SEQUENCE LISTING (10 sequences)

SEQ ID NO: 1 - protein, 121 aa, synthetic construct
EVQLVESGGGLVQPGGSLRLSCAASGFTFSSSWMNWVRQAPGKGLEWVGRIYPGDGDTNYNVKFKGRFTISRDDSKNSLYLQMNSLKTEDTAVYYCARSGFITTVRDFDYWGQGTLVTVSS

SEQ ID NO: 2 - protein, 5 aa, synthetic construct
SSWMN

SEQ ID NO: 3 - protein, 17 aa, synthetic construct
RIYPGDGDTNYNVKFKG

SEQ ID NO: 4 - protein, 12 aa, synthetic construct
SGFITTVRDFDY

SEQ ID NO: 5 - protein, 111 aa, synthetic construct
EIVLTQSPDFQSVTPKEKVTITCRASESVDTFGISFMNWFQQKPDQSPKLLIHEASNQGSGVPSRFSGSGSGTDFTLTINSLEAEDAATYYCQQSKEVPFTFGGGTKVEIK

SEQ ID NO: 6 - protein, 15 aa, synthetic construct
RASESVDTFGISFMN

SEQ ID NO: 7 - protein, 7 aa, synthetic construct
EASNQGS

SEQ ID NO: 8 - protein, 9 aa, synthetic construct
QQSKEVPFT

SEQ ID NO: 9 - protein, 451 aa, synthetic construct
EVQLVESGGGLVQPGGSLRLSCAASGFTFSSSWMNWVRQAPGKGLEWVGRIYPGDGDTNYNVKFKGRFTISRDDSKNSLYLQMNSLKTEDTAVYYCARSGFITTVRDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 218 aa, synthetic construct
EIVLTQSPDFQSVTPKEKVTITCRASESVDTFGISFMNWFQQKPDQSPKLLIHEASNQGSGVPSRFSGSGSGTDFTLTINSLEAEDAATYYCQQSKEVPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0301058 A1
(43) Pub. Date: Sep. 12, 2024

(54) INEBILIZUMAB AND METHODS OF USING THE SAME IN THE TREATMENT OR PREVENTION OF IGG4-RELATED DISEASE

(71) Applicant: Viela Bio, Inc., Deerfield, IL (US)

(72) Inventors: Jorn DRAPPA, La Jolla, CA (US); Eliezer KATZ, Durham, NC (US); Daniel M. CIMBORA, Glenwood, MD (US); Judith FALLOON, Bethesda, MD (US)

(21) Appl. No.: 18/431,078

(22) Filed: Feb. 2, 2024

Related U.S. Application Data
(63) Continuation of application No. PCT/US2022/074830, filed on Aug. 11, 2022.
(60) Provisional application No. 63/232,131, filed on Aug. 11, 2021.
(60) Provisional application No. 63/285,297, filed on Dec. 2, 2021.

(51) Int. Cl. C07K 16/28; A61K 31/573; A61K 39/00; A61P 37/02

(52) U.S. Cl. CPC C07K 16/2803; A61K 31/573; A61P 37/02; A61K 2039/545

(57) ABSTRACT

Provided herein are compositions and methods comprising anti-CD19 binding agents and methods of using the same for the treatment or amelioration of Immunoglobulin G4-related disease (IgG4-RD). Also provided are compositions and methods of using Inebilizumab for the treatment or amelioration of IgG4-RD and IgG4-RD flare.

### CROSS-REFERENCE TO RELATED APPLICATIONS

This application is a continuation of International Application No. PCT/US2022/074830, filed Aug. 11, 2022, which claims priority to U.S. Provisional Patent Application No. 63/232,131, filed Aug. 11, 2021, and U.S. Provisional Patent Application No. 63/285,297, filed Dec. 2, 2021, each of which is entirely incorporated herein by reference for all purposes.

### REFERENCE TO AN ELECTRONIC SEQUENCE LISTING

The contents of the electronic sequence listing (HOPA_035_02US_SeqList_ST26.xml; Size: 10,635 bytes; and Date of Creation: Jan. 23, 2024) are herein incorporated by reference in its entirety.

### TECHNICAL FIELD

The present disclosure is related to compositions and methods of treating or preventing IgG4 related disease comprising administering an anti-CD19 antibody to a subject in need.

### BACKGROUND

There are currently no medicinal products approved for the treatment of immunoglobulin G4-related disease (IgG4-RD), a rare disease. The majority of cases follow a relapsing course that can lead to permanent tissue damage with attendant morbidity and potential mortality. Glucocorticoids (GCs) are widely and effectively used for acute treatment of initial disease activity and of recurrent episodes (flares), but GCs do not prevent recurrence of active disease during their taper or after their discontinuation. Moreover, GCs are associated with substantial toxicity.

Thus, there is a high unmet medical need for therapies that prevent disease recurrence and limit GC exposure.

### BRIEF SUMMARY

Provided herein are methods of treating Immunoglobulin G4-related disease (IgG4-RD), comprising administering to a subject in need thereof an effective amount of Inebilizumab, wherein the administering is sufficient to reduce or eliminate an IgG4-RD flare, thereby treating the IgG4-RD. In embodiments, the administering is sufficient to reduce the IgG4-RD flare, as determined by: (a) reduced incidence of the IgG4-RD flare; (b) increased time to the IgG4-RD flare; or (c) a. and b. In embodiments, the incidence of the IgG4-RD flare is reduced by at least about 5%, 10%, 20%, 30%, 40%, 50%, 60%, 65%, 70%, 80%, 90%, or 100% as compared to an otherwise comparable subject lacking the administering.

In embodiments, the time to IgG4-RD flare is increased by at least about 3 days, 5 days, 7 days, 10 days, 14 days, 20 days, 1 month, 2 months, 3 months, 4 months, 6 months, 8 months, 10 months, 1 year, 2 years, 3 years, 4 year or 5 years, as compared to an otherwise comparable subject lacking the administering. In embodiments, the method comprises a. and b. In embodiments, the subject in need thereof was previously administered a glucocorticoid. In embodiments, the administering is effective in preventing new glucocorticoid administration in the subject in need thereof. In embodiments, the administering is effective in preventing increased glucocorticoid administration in the subject in need thereof.

Provided are methods of treating Immunoglobulin G4-related disease (IgG4-RD), comprising administering to a subject in need thereof an effective amount of Inebilizumab, wherein the administering is sufficient to extend time to detection of a IgG4-RD flare by at least 3 days as compared to an otherwise comparable subject lacking the administering. In embodiments, the time to detection of the IgG4-RD flare is extended by at about 1 month, 2 months, 3 months, 4 months, 6 months, 8 months, 10 months, 1 year, 2 years, 3 years, 4 year or 5 years.

Provided are methods of reducing the incidence of a flare in a subject having Immunoglobulin G4-related disease (IgG4-RD), the method comprising administering to the subject an effective amount of an anti-CD19 binding agent, thereby reducing the incidence of the flare in the subject. In embodiments, the anti-CD19 binding agent is effective in reducing or eliminating at least one of CD19 positive: B cells, plasmablasts, plasma cells, or any combination thereof. In embodiments, the anti-CD19 binding agent is an anti-CD19 antibody. In embodiments, the anti-CD19 antibody is Inebilizumab. In embodiments, the administering is effective in preventing administration of: a secondary immunotherapy, a corticosteroid, or a steroid-sparing agent. In embodiments, the method comprises the secondary immunotherapy, wherein the secondary immunotherapy comprises an antibody. In embodiments, the antibody is rituximab. In embodiments, the method comprises the corticosteroid, wherein the corticosteroid comprises prednisone. In embodiments, the method comprises the steroid-sparing agent, wherein the steroid-sparing agent is selected from the group consisting of: azathioprine and mycophenolate mofetil. In embodiments, the IgG4-RD flare is determined by one or more of: an IgG4-RD symptom, a physical exam manifestation, a laboratory result, an imaging result, or a biopsy result. In embodiments, the method comprises the IgG-RD symptom, wherein the IgG-RD symptom is selected from the group consisting of: headache, vision abnormality, salivary gland pain, lymph node swelling, pain, edema, dyspnea, cough, weight loss, itching, fatigue, rash, and combinations thereof. In embodiments, the method comprises the vision abnormality, wherein the vision abnormality is selected from the group consisting of: diplopia, proptosis, foreign body sensation, eye pain, vision blurring, vision loss, scleritis, eye numbness, tearing, crusting, redness, lacrimal gland swelling, and combinations thereof. In embodiments, the method comprises the physical exam manifestation, wherein the physical exam manifestation is selected from the group consisting of: central nervous system palsy, radiculomyelopathy, proptosis, supra-orbital swelling, peri-orbital swelling, field cuts, cranial nerve palsies, extraocular movement abnormality, infra-orbital/supra-orbital nerve enlargement, lacrimal gland swelling, salivary gland swelling, lymphadenopathy, increased respiratory rate, dry crackles, pleural effusion, palpable mass, edema, fibrosing mediastinitis, abdominal tenderness, jaundice, weight loss, abdominal tenderness, hyperpigmentation, erythematous nodules, and combinations thereof. In embodiments, the method comprises the laboratory result, wherein the laboratory result is selected from the group consisting of: cerebrospinal fluid pleocytosis, cerebrospinal fluid increased protein, pituitary endocrine dysfunction, pulmonary function abnormality, elevated creatinine, decreased estimated glomerular filtration rate (eGFR), elevated bilirubin, elevated alkaline phosphatase, elevated gamma-glutamyl transferase, elevated amylase, elevated lipase, low fecal elastase, high glucose, elevated alanine aminotransferase, elevated aspartate transaminase, hematuria, proteinuria, and combinations thereof. In embodiments, the method comprises the imaging result, wherein the imaging result is selected from the group consisting of: meningeal enhancement, pituitary mass, enlargement of extra-ocular muscle, enlargement of optic nerve, lacrimal gland swelling, salivary gland swelling, lymphadenopathy, pulmonary mass, pulmonary infiltrate, pulmonary fibrosis, pleural effusion, pleural thickening, peribronchovascular thickening, septal thickening, paravertebral mass, aneurysm, vascular thickening, ureteral stenosis, ureteral hydronephrosis, inflammation in retroperitoneum, inflammation in mesentery, inflammation in mediastinum, pancreatic mass, pancreatic enlargement, bile duct abnormality, pancreatic duct stricture, kidney enlargement, renal atrophy, pelvis thickening, and combinations thereof. In embodiments, the method comprises the biopsy result, wherein the biopsy result is selected from the group consisting of: pachymeningeal flare, pituitary gland flare, orbital flare, lacrimal gland flare, salivary gland flare, lymph node flare, lung flare, blood vessel flare, retroperitoneum flare, mediastinum flare, mesentery flare, pancreatic flare, bile duct flare, kidney flare, skin disease flare, and combinations thereof. In embodiments, from about 200-400 mg of the Inebilizumab is administered. In embodiments, from about 250-350 mg of the Inebilizumab is administered. In embodiments, about 300 mg of the Inebilizumab is administered. In embodiments, two weeks prior to administering the 300 mg Inebilizumab every 6 months, an initial 300 mg Inebilizumab dose is administered to the subject. In embodiments, the administering is intravenous. In embodiments, a method comprises: a second administering of the Inebilizumab, a third administering of the Inebilizumab, or both a second and third administering of the Inebilizumab. In embodiments, each of the administering, the second administering, or the third administering are separated by at least about 2 weeks, 1 month, 2 months, 3 months, 4 months, 5 months, 6 months, 8 months, 10 months, or up to about 1 year.

In aspects, pachymeningitis flare may be present when the subject shows new or worsening symptoms or physical examination consistent with IgG4-RD pachymeningitis in combination with (a) a CSF detection consistent with IgG4-RD pachymeningitis or with (b) an imaging finding confirming new or worsening meningeal involvement.

Provided are methods of treating Immunoglobulin G4-related disease (IgG4-RD), comprising administering to a subject in need thereof: (a) a pharmaceutical composition that comprises a corticosteroid in an amount sufficient to reduce an immune response in the subject; and (b) a pharmaceutical composition that comprises Inebilizumab in an amount sufficient to reduce a level of at least one of CD19 positive B cells, plasmablasts, plasma cells, or any combination thereof in the subject in need thereof, thereby treating the IgG4-RD. In embodiments, the corticosteroid is a glucocorticoid. In embodiments, the glucocorticoid is selected from the group consisting of: prednisone, methylprednisolone, hydrocortisone, betamethasone, dexamethasone, and combinations thereof. In embodiments, the glucocorticoid is prednisone. In embodiments, from about 1 mg/day to about 40 mg/day of the corticosteroid is administered. In embodiments, from about 5 mg/day to about 20 mg/day of the corticosteroid is administered. In embodiments, the corticosteroid administering is tapered. In embodiments, the tapering comprises an administering of 20 mg/day, 15 mg/day, 10 mg/day, and 5 mg/day. In embodiments, the tapering is completed over a period up to about 1 month, 2 months, 3 months, 4 months, or 5 months. In embodiments, the tapering is completed over a period of about 2 months. In embodiments, the reduced immune response comprises at least a 1-fold decrease in: a level of leukocyte migration to a site of inflammation, capillary permeability, inflammation, and any combination thereof. In embodiments, the Inebilizumab reduces the level of the at least one of CD19 positive B cells, plasmablasts, plasma cells, or any combination thereof by at least 1-fold, 5-fold, 10-fold, 30-fold, 60-fold, 90-fold, 150-fold, 200-fold, or 300-fold as compared to an otherwise comparable subject lacking the administering. In embodiments, after the tapering, a reduction of IgG4-RD flare is observed as determined by: (a) reduced incidence of the IgG4-RD flare; (b) increased time to the IgG4-RD flare; or (c) a. and b. In embodiments, a method comprises a., wherein the incidence of the IgG4-RD is reduced by at least about 10%, 20%, 30%, 40%, 50%, 60%, 65%, 70%, 80%, 90%, or 100% as compared to an otherwise comparable subject lacking the administering. In embodiments, a method comprises b., wherein the time to IgG4-RD flare is increased by at least about 3 days, 5 days, 7 days, 10 days, 14 days, 20 days, 1 month, 2 months, 3 months, 4 months, 6 months, 8 months, 10 months, 1 year, 2 years, 3 years, 4 year or 5 years as compared to an otherwise comparable subject lacking the administering. In embodiments, a method comprises a. and b. In embodiments, a method comprises determining a level of a marker in a sample of the subject in need thereof, wherein the marker is from a class selected from the group consisting of: immunoglobulin, complement, cellular, serum, RNA, DNA, and combinations thereof. In embodiments, the IgG4-RD flare is determined by one or more of: an IgG4-RD symptom, a physical exam manifestation, a laboratory result, an imaging result, a biopsy result, or a combination thereof. In embodiments, a method comprises the IgG-RD symptom, wherein the IgG-RD symptom is selected from the group consisting of: headache, vision abnormality, salivary gland pain, lymph node swelling, Dyspnea, pain, edema, dyspnea, cough, weight loss, itching, fatigue, rash, and combinations thereof. In embodiments, a method comprises the vision abnormality, wherein the vision abnormality is selected from the group consisting of: Diplopia, proptosis, foreign body sensation, eye pain, vision blurring, vision loss, scleritis, eye numbness, tearing, crusting, redness, lacrimal gland swelling, and combinations thereof. In embodiments, a method comprises the physical exam manifestation, wherein the physical exam manifestation is selected from the group consisting of: central nervous system palsy, radiculomyelopathy, proptosis, supra-orbital swelling, peri-orbital swelling, field cuts, cranial nerve palsies, extraocular movement abnormality, infra-orbital/supra-orbital nerve enlargement, lacrimal gland swelling, salivary gland swelling, lymphadenopathy, increased respiratory rate, dry crackles, pleural effusion, palpable mass, edema, fibrosing mediastinitis, abdominal tenderness, jaundice, weight loss, abdominal tenderness, hyperpigmentation, erythematous nodules, and combinations thereof. In embodiments, a method comprises the laboratory result, wherein the laboratory result is selected from the group consisting of: cerebrospinal fluid pleocytosis, cerebrospinal fluid increased protein, pituitary endocrine dysfunction, pulmonary function abnormality, elevated creatinine, decreased estimated glomerular filtration rate (eGFR), elevated bilirubin, elevated alkaline phosphatase, elevated gamma-glutamyl transferase, elevated amylase, elevated lipase, low fecal elastase, high glucose, elevated alanine aminotransferase, elevated aspartate transaminase, hematuria, proteinuria, and combinations thereof. In embodiments, a method comprises the imaging result, wherein the imaging result is selected from the group consisting of: meningeal enhancement, pituitary mass, enlargement of extra-ocular muscle, enlargement of optic nerve, lacrimal gland swelling, salivary gland swelling, lymphadenopathy, pulmonary mass, pulmonary infiltrate, pulmonary fibrosis, pleural effusion, pleural thickening, peribronchovascular thickening, septal thickening, paravertebral mass, aneurysm, vascular thickening, ureteral stenosis, ureteral hydronephrosis, inflammation in retroperitoneum, inflammation in mesentery, inflammation in mediastinum, pancreatic mass, pancreatic enlargement, bile duct abnormality, pancreatic duct stricture, kidney enlargement, renal atrophy, pelvis thickening, and combinations thereof. In embodiments, a method comprises the biopsy result, wherein the biopsy result is selected from the group consisting of: pachymeningeal flare, pituitary gland flare, orbital flare, lacrimal gland flare, salivary gland flare, lymph node flare, lung flare, blood vessel flare, retroperitoneum flare, mediastinum flare, mesentery flare, pancreatic flare, bile duct flare, kidney flare, skin disease flare, and combinations thereof. In embodiments, a method comprises administering an antihistamine, an antipyretic, or both. In embodiments, a method comprises the antihistamine, wherein the antihistamine is diphenhydramine. In embodiments, a method comprises the antipyretic, wherein the antipyretic is acetaminophen. In embodiments, the antihistamine, antipyretic, or both are administered prior to the pharmaceutical composition that comprises Inebilizumab.

Provided are methods of treating Immunoglobulin G4-related disease (IgG4-RD), the method comprising: administering Inebilizumab to a patient in need of treatment for IgG4-RD, wherein the Inebilizumab is administered intravenously at a dose of 300 mg every 6 months. In embodiments, two weeks prior to administering the 300 mg Inebilizumab every 6 months, an initial 300 mg Inebilizumab dose is administered to the subject.

There are two identified subsets of IgG4-RD patients. The methods disclosed herein are suitable for either subtype. The first type is called a proliferative subtype. A proliferative subtype patient is characterized by one or more of the following: glandular or epithelial tissue origin of disease, high frequency of atopy, multiple organs are typically affected (for example, one or more of lymph nodes, lacrimal gland, major salivary glands, pancreas, bile ducts, kidney, lung pituitary and paranasal sinus. Proliferative subtypes patients typical have high levels of IgG4, IgG1 and IgE. Eosinophilia and hypocomplementemia are also common. They may also show produce autoantibodies to one or more of laminin-511, galectin-3, annexin A11, and prohibitin. In lymphoplasmacytic infiltrates they may exhibit over 50 or over 100 IgG4-positive cells per HPF.

The second subtype is called a fibrotic subtype. Fibrotic subtype patients show an origin of disease from extra glandular tissues rather than distinct organs. Fibrotic subtypes patient typical show slight elevation or normal levels of IgG4, IgG1 and IgE. Germinal center pathology is unusual. In lymphoplasmacytic infiltrates the fibrotic subtype patient may fewer than 50 IgG4-positive cells per HPF. Obliterative phlebitis is common. Germinal center pathology and storiform fibrosis are also common. Multiple organs are typically affected (for example, one or more of retroperitoneum, aorta and periaortic tissue, mesentery, pachymeninges, thyroid, and mediastinum.

In aspects, the IgG4-RD patient does not have one or more of the following: prominent neutrophilic infiltrate, necrotizing vasculitis, prominent necrosis, primarily granulomatous inflammation.

Reducing flare incidence is particularly advantageous in these patients. The incidence of flares is about 35% annually; however, it can be about 20%, about 30%, about 40% or about 50%. The methods disclosed herein reduce the risk of flare by about 40%, about 50%, about 60%, about 65% or about 70%. Thus, an individual having a 35% incidence rate would have, on average, about 1 attack every 3 years. The therapies would

Provided are methods of treating a patient diagnosed with IgG4-RD, the method comprising: administering Inebilizumab to a patient diagnosed with IgG4-RD, wherein the Inebilizumab is administered intravenously with a first initial dose of 300 mg, a second initial dose of 300 mg two weeks after the first initial dose, and subsequent doses of 300 mg every 6 months following the first initial dose.

These and other embodiments are described below.

### BRIEF DESCRIPTION OF THE DRAWINGS

The accompanying figures, which are incorporated herein and form a part of the specification, illustrate some, but not the only or exclusive, example embodiments and/or features. It is intended that the embodiments and figures disclosed herein are to be considered illustrative rather than limiting.

FIG. 1 shows an exemplary study design.

FIG. 2A provides Inebilizumab VH (SEQ ID NO:1) amino acid sequence and FIG. 2B provides Inebilizumab VL (SEQ ID NO:5) amino acid sequence. The amino acid sequence of each of Inebilizumab VH CDR1 (SEQ ID NO:2), VH CDR2 (SEQ ID NO:3), VH CDR3 (SEQ ID NO:4), VL CDR1 (SEQ ID NO:6), VL CDR2 (SEQ ID NO:7) and VL CDR3 (SEQ ID NO:8) is separately indicated within its respective VH and VL amino acid sequence.

FIG. 3 shows a schematic of an exemplary flare assessment flow chart. AC=Adjudication committee; IgG4-RD=immunoglobulin G4-related disease. The schematic outlines the assessment and adjudication process for suspected flares and highlights the events that may contribute to the primary endpoint.

### DETAILED DESCRIPTION

The following description includes information that may be useful in understanding the present disclosure. It is not an admission that any of the information provided herein is prior art or relevant to the presently claimed disclosures, or that any publication specifically or implicitly referenced is prior art.

The pathogenesis of IgG4-RD suggests that B-cell depletion may be an effective avenue for therapeutic intervention. An important role for B cells, particularly plasmablasts and plasma cells, in the pathogenesis of the disease appears likely. The anti CD19 B cell-depleting activity of Inebilizumab suggests that it may provide benefit as treatment for IgG4-RD. Accordingly, provided herein are compositions and methods of using the same for the prevention of flare of IgG4-RD and treatment thereof.

### Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art.

As used herein, the singular forms “a.” “an,” and “the: include plural referents unless the content clearly dictates otherwise.

The term “about” or “approximately” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

As used herein, the terms “antibody” and “antibodies” (immunoglobulins) encompass monoclonal antibodies (including full-length monoclonal antibodies), polyclonal antibodies, multispecific antibodies (e.g., bispecific antibodies) formed from at least two intact antibodies, human antibodies, humanized antibodies, camelised antibodies, chimeric antibodies, single-chain Fvs (scFv), single-chain antibodies, single domain antibodies, domain antibodies, Fab fragments, F(ab′)2 fragments, antibody fragments that exhibit the desired biological activity, disulfide-linked Fvs (sdFv), and anti-idiotypic (anti-Id) antibodies (including, e.g., anti-Id antibodies to antibodies of the invention), intrabodies, and epitope-binding fragments of any of the above. In particular, antibodies include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

When referring to a nucleic acid sequence or protein sequence, the term “identity” is used to denote similarity between two sequences. Unless otherwise indicated, percent identities described herein are determined using the BLAST algorithm available at the world wide web address: blast.ncbi.nlm.nih.gov/Blast.cgi using default parameters.

A “therapeutically effective” amount as used herein is an amount that provides some improvement or benefit to the subject. Stated in another way, a “therapeutically effective” amount is an amount that provides some alleviation, mitigation, and/or decrease in at least one clinical symptom. Clinical symptoms associated with the disorders that can be treated by the methods of the invention are well-known to those skilled in the art. Further, those skilled in the art will appreciate that the therapeutic effects need not be complete or curative, as long as some benefit is provided to the subject.

### Immunoglobulin G4-Related Disease (IgG4-RD)

Immunoglobulin G4-related disease (IgG4-RD) is a chronic, relapsing remitting, immune-mediated fibroinflammatory disorder that can affect virtually every organ system, with a predilection for salivary glands, orbits, lacrimal glands, pancreas, biliary tree, lungs, kidneys, aorta and retroperitoneum, meninges, and thyroid gland. Pancreato-hepatobiliary disease is present in approximately half of cases, and disease is present in at least 2 organs in approximately three-fourths of patients. Organ involvement generally manifests as swelling, presence of a mass, or organ-specific consequences of tissue damage. The clinical presentation is generally subacute, and most patients are neither acutely ill nor febrile; however, weight loss can be significant, particularly in the setting of exocrine pancreatic failure in IgG4-related autoimmune pancreatitis. Multi-organ involvement is common either at baseline or after progression over time. The course depends in part on the predominant organ affected. For example, IgG4-RD-related cholangitis can lead to hepatic failure within months; autoimmune pancreatitis leads to diabetes mellitus, exocrine insufficiency, or both; tubulointerstitial nephritis can lead to renal failure; and aortitis can lead to aneurysms and dissections. The majority of afflicted subjects are men, and most are older than 50 years of age. In a study from Japan, the incidence of IgG4-RD was estimated to be 2.8-10.8/million population, with a median age of onset of 58 years.

In embodiments, manifestations of IgG4-RD are linked by a common histopathology: a dense lymphoplasmacytic infiltrate containing IgG4-positive (IgG4+) plasma cells, storiform fibrosis, and obliterative phlebitis, often accompanied by tissue eosinophilia. The inflammatory infiltrate is a mixture of T and B lymphocytes, with B cells typically organized in germinal centers. Although all Ig subclasses may be present, IgG4 predominates, and the IgG4+: IgG+ plasma cell ratio is an important feature.

The IgG4 antibody produced by plasmablasts and plasma cells in IgG4-RD is probably not pathogenic. However, there is a general correlation between IgG4 serum concentration and disease severity/extent; most patients have elevated IgG4 levels that decline with clinical response to GC treatment.

In embodiments, symptomatic patients with IgG4-RD may require treatment. Glucocorticoids (GCs) are the cornerstone of therapy, and rapid GC responsiveness, particularly for the proliferative subtype, is a hallmark of the disease. Treatment has not been fully standardized, but international consensus guidelines recommend 2-4 weeks of induction with 0.6 mg/kg of prednisone equivalent (˜30-40 mg/day) tapering over 8-12 weeks. Despite remission occurring in the majority of patients, disease flares requiring re-institution of therapy are typical. Patients with more fibrosis in the affected organ tend to have worse clinical responses to treatment; in contrast to proliferative disease, fibrotic disease tends not to improve with treatment.

In embodiments, steroid-sparing drugs such as disease-modifying anti-rheumatic drugs (DMARDs) may be utilized. In embodiments, B-cell depletion with the cluster of differentiation (CD)20-targeted agent rituximab may be utilized.

### IgG4-RD Flare

In embodiments, an IgG4-RD flare comprises new or worsening signs and symptoms of IgG4-RD disease activity that meet one or more organ-specific flare criteria. In embodiments, there may be no clear alternative diagnosis or conflicting biopsy findings.

In embodiments, an IgG4-RD flare comprises involvement of the pachymeninges. In embodiments, a subject in need thereof may comprise a headache. In embodiments, a physical exam may reveal palsy of the central nervous system, neurologic abnormalities consistent with radiculomyelopathy, or a combination thereof. In embodiments a laboratory exam may reveal CSF pleocytosis and/or CSF increased protein. In embodiments imaging may reveal Meningeal enhancement or thickening, in embodiments, a biopsy may be consistent with a pachymeninges flare.

In embodiments, an IgG4-RD flare comprises involvement of the pituitary gland. In embodiments, a subject in need thereof may comprise a visual field abnormality, headache, symptoms consistent with anterior or posterior pituitary endocrine failure, and combinations thereof. In embodiments, a physical exam may reveal CN palsy, visual field abnormalities, or a combination thereof. In embodiments a laboratory exam may reveal pituitary endocrine dysfunction. In embodiments imaging may reveal a pituitary mass or enhancement. In embodiments, a biopsy may be consistent with a pituitary gland flare.

In embodiments, an IgG4-RD flare comprises involvement of one or both orbits. In embodiments, a subject in need thereof may comprise diplopia, proptosis, foreign body sensation, eye or retrobulbar discomfort or pain, or other visual symptoms including vision blurring or loss, symptoms consistent with scleritis, symptoms from compression of peripheral nerves in the area of the orbit, such as trigeminal and infra-orbital nerves (pain or numbness), and combinations thereof. In embodiments, a physical exam may reveal Proptosis, supra-orbital swelling or other peri-orbital swelling consistent with enlargement of extra-ocular muscles, field cuts, cranial nerve palsies, extraocular movement abnormality, infra-orbital/supra-orbital nerve enlargement, or a combination thereof. In embodiments a laboratory exam may reveal pituitary endocrine dysfunction. In embodiments imaging may reveal orbital disease (enlargement of extra-ocular muscles, enlargement of optic nerve including abnormalities of retrobulbar space or within cavernous sinus). In embodiments, a biopsy may be consistent with orbital flare.

In embodiments, an IgG4-RD flare comprises involvement of one or both lacrimal glands. In embodiments, a subject in need thereof may comprise lacrimal gland discomfort, pain or swelling; redness of the eye, excessive tearing; eyelid crusting: blurred vision, and combinations thereof. In embodiments, a physical exam may reveal lacrimal gland swelling and/or a lacrimal gland mass. In embodiments imaging may be consistent with lacrimal gland swelling. In embodiments, a biopsy may be consistent with lacrimal gland flare.

In embodiments, an IgG4-RD flare comprises involvement of one or more salivary glands. In embodiments, a subject in need thereof may comprise pain, swelling, or both pain and swelling in one or more salivary glands. In embodiments, a physical exam may reveal salivary gland swelling or tenderness. In embodiments imaging may be consistent with salivary gland swelling. In embodiments, a biopsy may be consistent with salivary gland flare.

In embodiments, an IgG4-RD flare comprises involvement of one or more lymph nodes. In embodiments, a subject in need thereof may comprise swelling in the one or more lymph nodes. In embodiments, a physical exam may reveal lymphadenopathy. In embodiments imaging may be consistent with Lymphadenopathy. In embodiments, a biopsy may be consistent with lymph node flare.

In embodiments, an IgG4-RD flare comprises involvement of the lungs. In embodiments, lung involvement comprises pleura, parenchyma, or both. In embodiments, a subject in need thereof may comprise dyspnea at rest or with exertion, cough, or both. In embodiments, a physical exam may reveal increased respiratory rate, pleural effusion, dry crackles compatible with pulmonary fibrosis, localized diminished breath sounds, findings consistent with infiltrate, or a combination thereof. In embodiments a laboratory exam may reveal new or worsening pulmonary function test abnormalities consistent with lung flare. In embodiments imaging may reveal pulmonary nodules or mass and/or pulmonary infiltrate/ground glass opacities consistent with interstitial pneumonia and/or pulmonary fibrosis and/or pleural effusion or pleural thickening and/or peribronchovascular and septal thickening and/or paravertebral mass, paravertebral band-like soft tissue in thorax. In embodiments, a biopsy may be consistent with lung flare.

In embodiments, an IgG4-RD flare comprises involvement of the heart. In embodiments, heart involvement comprises the aorta, large blood vessels, or both. In embodiments, a subject in need thereof may comprise pain, palpable mass which may be systemic or constitutional. In embodiments, a physical exam may reveal a palpable arterial mass. In embodiments, the mass may be pulsatile, or bruit. In embodiments imaging may reveal aneurysm, dissection, thickening/enhancement of vessel wall or other vessel abnormalities. In embodiments, a biopsy may be consistent with a heart flare.

In embodiments, an IgG4-RD flare comprises involvement of the retroperitoneum, mediastinum, and/or mesentery. In embodiments, a subject in need thereof may comprise pain (e.g., flank, back, thighs, abdominal, other including chronic pain), leg edema, dyspnea, cough. In embodiments, a physical exam may reveal a palpable mass or findings consistent with superior vena cava syndrome, leg edema, or fibrosing mediastinitis. In embodiments a laboratory exam may reveal retroperitoneal involvement of ureters: elevated creatinine, decreased estimated glomerular filtration rate (eGFR). In embodiments imaging may reveal mass lesion, ureteral stenosis or hydronephrosis, findings consistent with superior vena cava syndrome, other evidence of inflammation in retroperitoneum typically with enhancement (often infra-renal, peri-aortic distribution extending down to iliac vessels but may involve root of mesentery), circumferential/antero-lateral soft tissue around infrarenal aorta or iliac arteries, other radiologic evidence of inflammation in mesentery or mediastinum. In embodiments, a biopsy may be consistent with retroperitoneum, mediastinum, and/or mesentery flare.

In embodiments, an IgG4-RD flare comprises involvement of the pancreas. In embodiments, pancreas involvement comprises common bile duct involvement. In embodiments, a subject in need thereof may comprise pain (e.g., flank, back, abdominal), and/or weight loss. In embodiments, a physical exam may reveal abdominal tenderness, jaundice, palpable mass, weight loss. In embodiments a laboratory exam may reveal elevated bilirubin, alkaline phosphatase, GGT, amylase and/or lipase: other, including low fecal elastase, high glucose/HbA1C. In embodiments imaging may reveal pancreatic mass or diffuse pancreatic enlargement with loss of lobulations, diffuse pancreatic enlargement, pseudo capsule, pancreatic duct stricture, common bile duct abnormality. In embodiments, a biopsy may be consistent with pancreatic/common bile duct flare.

In embodiments, an IgG4-RD flare comprises involvement of the biliary tree. In embodiments, biliary tree involvement comprises IgG4-RD sclerosing cholangitis. In embodiments, a subject in need thereof may comprise itching, abdominal pain, right upper quadrant pain. In embodiments, a physical exam may reveal abdominal tenderness, jaundice, right upper quadrant fullness, or any combination thereof. In embodiments a laboratory exam may reveal elevated: bilirubin, ALT/AST, alkaline phosphatase, GGT. In embodiments imaging may reveal thickening, mass, strictures, dilatation of extra-hepatic and/or intrahepatic bile ducts. In embodiments, a biopsy may be consistent with bile duct/biliary tree flare.

In embodiments, an IgG4-RD flare comprises kidney involvement. In embodiments, a subject in need thereof may comprise fatigue, and/or mental status changes. In embodiments, a physical exam may reveal edema. In embodiments a laboratory exam may reveal one or more of elevated creatinine, decreased eGFR, hematuria, proteinuria, hypocomplementemia, or combinations thereof. In embodiments imaging may reveal diffuse kidney enlargement, renal abnormality including, hypodense lesions in the renal cortex, renal atrophy, and/or pelvis thickening. In embodiments, a biopsy may be consistent with kidney flare.

In embodiments, an IgG4-RD flare comprises skin involvement. In embodiments, a subject in need thereof may comprise a rash. In embodiments, a physical exam may reveal erythematous papules or nodules, hyperpigmented lesions, other skin lesions, or combinations thereof. In embodiments, a biopsy may be consistent with skin flare.

In embodiments, an IgG4-RD flare comprises thyroid involvement. In embodiments, a subject in need thereof has Riedel Thyroiditis. In embodiments, a subject with Riedel Thyroiditis has secondary involvement of additional tissues. In embodiments, a subject with IgG4-RD flare may have affected tissues exhibiting sclerosis and/or mass formation. Additional tissues may comprise one or more of liver, breast, prostate, maxillary sinus, nasal septum, pericardium, peripheral nerves, and the like. In embodiments secondary involvement of additional tissues results in one or more of retroperitoneal fibrosis, pancreatic fibrosis, mediastinal fibrosis, orbital pseudotumor, and sclerosing cholangitis.

In embodiments, administration of Inebilizumab is sufficient to reduce or eliminate an IgG4-RD flare. In embodiments, administration of Inebilizumab is sufficient to reduce a level of any one of the aforementioned symptoms, laboratory exams, physical exams, imaging results, and/or biopsies as compared to an otherwise comparable method lacking the administering. In embodiments, administration of Inebilizumab is sufficient to eliminate an IgG4-RD flare. In embodiments, the administering of Inebilizumab is sufficient to reduce an IgG4-RD flare, as determined by (a) reduced incidence of the IgG4-RD flare: (b) increased time to the IgG4-RD flare; or both (a) and (b).

In embodiments, incidence to IgG4-RD flare is the date of detection of flare, or initiation of any flare treatment. Flare treatment comprises any one of: new or increased glucocorticoid treatment, other immunotherapy, interventional procedure, or any combination thereof. In embodiments, administration of Inebilizumab is sufficient to reduce incidence of an IgG4-RD flare by at least about or at most about: 1-fold, 2-fold, 4-fold, 6-fold, 8-fold, 10-fold, 12-fold, 14-fold, 16-fold, 18-fold, 20-fold, 22-fold, 24-fold, 26-fold, 28-fold, 30-fold, 32-fold, 34-fold, 36-fold, 38-fold, 40-fold, 42-fold, 44-fold, 46-fold, 48-fold, 50-fold, 52-fold, 54-fold, 56-fold, 58-fold, 60-fold, 62-fold, 64-fold, 66-fold, 68-fold, 70-fold, 72-fold, 74-fold, 76-fold, 78-fold, 80-fold, 82-fold, 84-fold, 86-fold, 88-fold, 90-fold, 92-fold, 94-fold, 96-fold, 98-fold, 100-fold, 102-fold, 104-fold, 106-fold, 108-fold, 110-fold, 112-fold, 114-fold, 116-fold, 118-fold, 120-fold, 122-fold, 124-fold, 126-fold, 128-fold, 130-fold, 132-fold, 134-fold, 136-fold, 138-fold, 140-fold, 142-fold, 144-fold, 146-fold, 148-fold, 150-fold as compared to an otherwise comparable method lacking the administration. In embodiments, administration of Inebilizumab is sufficient to reduce incidence of an IgG4-RD flare by at least about or at most about: 5%, 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, or up to about 400% as compared to an otherwise comparable method lacking the administration.

In embodiments, administration of Inebilizumab is sufficient to increase time to an IgG4-RD flare by at least about 1-fold, 2-fold, 4-fold, 6-fold, 8-fold, 10-fold, 12-fold, 14-fold, 16-fold, 18-fold, 20-fold, 22-fold, 24-fold, 26-fold, 28-fold, 30-fold, 32-fold, 34-fold, 36-fold, 38-fold, 40-fold, 42-fold, 44-fold, 46-fold, 48-fold, 50-fold, 52-fold, 54-fold, 56-fold, 58-fold, 60-fold, 62-fold, 64-fold, 66-fold, 68-fold, 70-fold, 72-fold, 74-fold, 76-fold, 78-fold, 80-fold, 82-fold, 84-fold, 86-fold, 88-fold, 90-fold, 92-fold, 94-fold, 96-fold, 98-fold, 100-fold, 102-fold, 104-fold, 106-fold, 108-fold, 110-fold, 112-fold, 114-fold, 116-fold, 118-fold, 120-fold, 122-fold, 124-fold, 126-fold, 128-fold, 130-fold, 132-fold, 134-fold, 136-fold, 138-fold, 140-fold, 142-fold, 144-fold, 146-fold, 148-fold, 150-fold as compared to an otherwise comparable method lacking the administration. In embodiments, administration of Inebilizumab is sufficient to increase time to an IgG4-RD flare by at least about or at most about 1 day, 5 days, 10 days, 15 days, 20 days, 25 days, 30 days, 35 days, 40 days, 45 days, 50 days, 55 days, 60 days, 65 days, 70 days, 75 days, 80 days, 85 days, 90 days, 95 days, 100 days, 105 days, 110 days, 115 days, 120 days, 125 days, 130 days, 135 days, 140 days, 145 days, 150 days, 155 days, 160 days, 165 days, 170 days, 175 days, 180 days, 185 days, 190 days, 195 days, 200 days, 205 days, 210 days, 215 days, 220 days, 225 days, 230 days, 235 days, 240 days, 245 days, 250 days, 255 days, 260 days, 265 days, 270 days, 275 days, 280 days, 285 days, 290 days, 295 days, 300 days, 305 days, 310 days, 315 days, 320 days, 325 days, 330 days, 335 days, 340 days, 345 days, 350 days, 355 days, 360 days, or up to about 365 days. In embodiments, the time to IgG4-RD flare is increased by at least about 3 days, 5 days, 7 days, 10 days, 14 days, 20 days, 1 month, 2 months, 3 months, 4 months, 6 months, 8 months, 10 months, 1 year, 2 years, 3 years, 4 year or 5 years as compared to an otherwise comparable subject lacking the administering.

In embodiments, administration of Inebilizumab is sufficient to reduce incidence of an IgG4-RD flare as determined by a reduced level of symptoms in a subject. In embodiments, administration of Inebilizumab is sufficient to reduce incidence of an IgG4-RD flare as determined by reversion to normalcy of results of laboratory exams. In embodiments, administration of Inebilizumab is sufficient to reduce manifestations of an IgG4-RD flare as determined by reversion to normalcy of results of a physical exam. In embodiments, administration of Inebilizumab is sufficient to reduce manifestations of an IgG4-RD flare as determined by reversion to normalcy of results of imaging results. In embodiments, administration of Inebilizumab is sufficient to reduce manifestations of an IgG4-RD flare as determined by reversion to normalcy of results of biopsy. In embodiments, any of the aforementioned symptoms, laboratory exams, physical exams, imaging results, and/or biopsies may comprise increases or reductions in levels of about 1-fold, 2-fold, 4-fold, 6-fold, 8-fold, 10-fold, 12-fold, 14-fold, 16-fold, 18-fold, 20-fold, 22-fold, 24-fold, 26-fold, 28-fold, 30-fold, 32-fold, 34-fold, 36-fold, 38-fold, 40-fold, 42-fold, 44-fold, 46-fold, 48-fold, 50-fold, 52-fold, 54-fold, 56-fold, 58-fold, 60-fold, 62-fold, 64-fold, 66-fold, 68-fold, 70-fold, 72-fold, 74-fold, 76-fold, 78-fold, 80-fold, 82-fold, 84-fold, 86-fold, 88-fold, 90-fold, 92-fold, 94-fold, 96-fold, 98-fold, 100-fold, 102-fold, 104-fold, 106-fold, 108-fold, 110-fold, 112-fold, 114-fold, 116-fold, 118-fold, 120-fold, 122-fold, 124-fold, 126-fold, 128-fold, 130-fold, 132-fold, 134-fold, 136-fold, 138-fold, 140-fold, 142-fold, 144-fold, 146-fold, 148-fold, 150-fold, 152-fold, 154-fold, 156-fold, 158-fold, 160-fold, 162-fold, 164-fold, 166-fold, 168-fold, 170-fold, 172-fold, 174-fold, 176-fold, 178-fold, 180-fold, 182-fold, 184-fold, 186-fold, 188-fold, 190-fold, 192-fold, 194-fold, 196-fold, 198-fold, 200-fold, 202-fold, 204-fold, 206-fold, 208-fold, 210-fold, 212-fold, 214-fold, 216-fold, 218-fold, 220-fold, 222-fold, 224-fold, 226-fold, 228-fold, 230-fold, 232-fold, 234-fold, 236-fold, 238-fold, 240-fold, 242-fold, 244-fold, 246-fold, 248-fold, or up to about 250-fold as compared to an otherwise comparable subject lacking the administering of Inebilizumab.

### Inebilizumab and Anti-CD19 Binding Agents

In embodiments, provided herein are anti-CD19 binding agents. Anti-CD19 binding agents may be used in disclosed therapeutic regimens for the treatment of IgG4-RD. In embodiments, a therapeutic regimen comprising any of the disclosed anti-CD19 binding agents results in B-cell reduction or elimination in a subject in need thereof. B cells, particularly plasmablasts and plasma cells, contribute to disease thereby reducing or eliminating them is of benefit to subjects with IgG4-RD. In embodiments, an anti-CD19 binding agent is effective in reducing or eliminating at least one of CD19 positive: B cells, plasmablasts, plasma cells, or any combination thereof. In embodiments, an anti-CD19 binding agent comprises an anti-CD19 antibody. In embodiments, an anti-CD19 binding agent comprises Inebilizumab.

Inebilizumab (also known as MEDI-551) is a humanized, affinity-optimized, afucosylated IgG1 kappa monoclonal antibody (mAb) known as 16C-aFuc that binds to the B-cell specific surface antigen CD19, resulting in reduction or elimination of CD19+ B cells. Inebilizumab is glycoengineered by expression of mAb 16C4 in a fucosyltransferase deficient Chinese hamster ovary producer cell line (BioWa Potelligent® Technology), which generates a homogenously afucosylated antibody with enhanced antibody-dependent cellular cytotoxicity. In contrast to the anti-CD20 mAb rituximab, Inebilizumab does not mediate complement-dependent cytotoxicity but eliminates B cells via antibody-dependent cellular cytotoxicity and antibody-medicated cellular phagocytosis mechanisms.

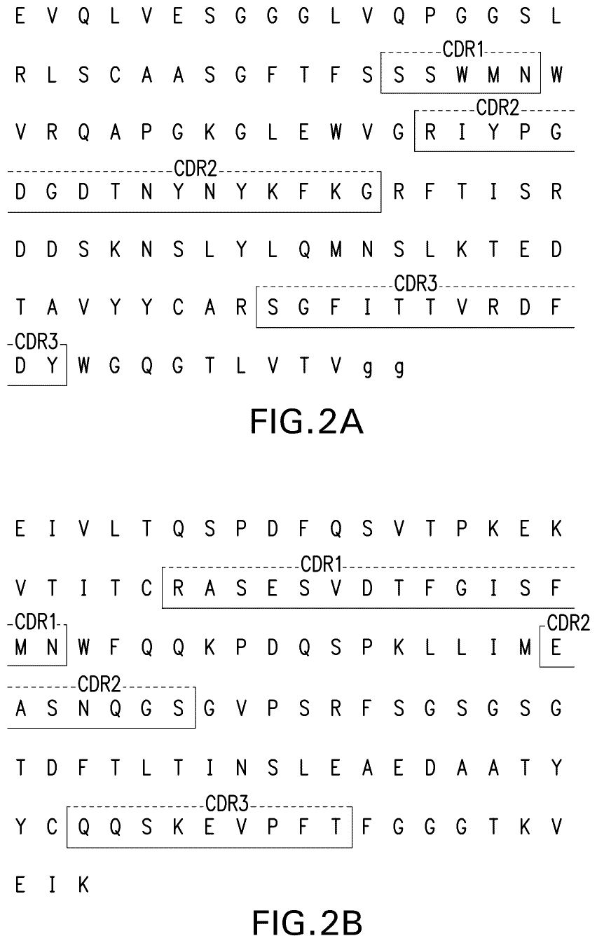

In embodiments, Inebilizumab comprises a sequence from Table 1. In embodiments, Inebilizumab may have the VH amino acid sequence and a VL amino acid sequence from Table 1. In embodiments. Inebilizumab or a derivative of Inebilizumab comprises at least about or at most about 50%, 55%, 60%, 65%, 70%, 75%, 80%, 85%, 90%, 95%, 96%, 97%, 98%, 99%, or up to about 100% identity with any one of SEQ ID NO: 1-SEQ ID NO: 10 shown in Table 1. In embodiments, any one of the sequences from Table 1 can be modified. In embodiments, a modification comprises one or more truncations, deletions, insertions, and combinations thereof. In embodiments, a modification does not alter the function of Inebilizumab. A modification can occur at any of the residues provided in Table 1 and in any number of residues from Table 1. In embodiments, a modification can comprise from 0-3, 0-5, 0-10, 0-20, 1-3, 1-5, 1-10, 1-20, 3-8, 3-10, 3-15, 5-8, 5-10, or 5-20 residues. In embodiments, a modification can occur in 0, 1, 2, 3, 4, 5, 6, 7, 8, 9, 10, 11, 12, 13, 14, 15, 20, 30, 40, 50, 60, 70, 80, 90, 100, 200, 300, 400, or 450 residues. In embodiments, all of the residues of Table 1 comprise a modification. A derivative of Inebilizumab includes but is not limited to an antibody with the VH amino acid sequence and the VL amino acid sequence as shown in FIG. 2A and FIG. 2B, but for one or more substitutions in amino acid residues that do not alter the function of Inebilizumab. In embodiments, an Inebilizumab derivative is an antibody with the VH amino acid sequence and the VL amino acid sequence as shown in FIG. 2A or FIG. 2B, with 1, 2, 3, 4, or 5 amino acid residue substitutions and/or deletions. In aspects, a derivative of Inebilizumab includes the same CDR amino acid sequences as the VH and the VL sequences as shown in FIG. 2A or FIG. 2B but may have one or more amino acid substitutions in the framework regions of the VH and the VL sequences.

TABLE 1
Exemplary Inebilizumab sequences

| SEQ ID | | |
|---|---|---|
| NO | Description | Sequence |
| 1 | Variable Heavy (VH) | EVQLVESGGGLVQPGGSLRLSCAASGFTFSSSWMN |
| | | WVRQAPGKGLEWVGRIYPGDGDTNYNVKFKGRFT |
| | | ISRDDSKNSLYLQMNSLKTEDTAVYYCARSGFITTV |
| | | RDFDYWGQGTLVTVSS |
| 2 | VH CDR1 | SSWMN |
| 3 | VH CDR2 | RIYPGDGDTNYNVKFKG |
| 4 | VH CDR3 | SGFITTVRDFDY |
| 5 | Variable Light (VL) | EIVLTQSPDFQSVTPKEKVTITCRASESVDTFGISFM |
| | | NWFQQKPDQSPKLLIHEASNQGSGVPSRFSGSGSGT |
| | | DFTLTINSLEAEDAATYYCQQSKEVPFTFGGGTKVE |
| | | IK |
| 6 | VL CDR1 | RASESVDTFGISFMN |
| 7 | VL CDR2 | EASNQGS |
| 8 | VL CDR3 | QQSKEVPFT |
| 9 | Heavy Chain | EVQLVESGGGLVQPGGSLRLSCAASGFTFSSSWMN |
| | | WVRQAPGKGLEWVGRIYPGDGDTNYNVKFKGRFT |
| | | ISRDDSKNSLYLQMNSLKTEDTAVYYCARSGFITTV |
| | | RDFDYWGQGTLVTVSSASTKGPSVFPLAPSSKSTSG |
| | | GTAALGCLVKDYFPEPVTVSWNSGALTSGVHTFPA |
| | | VLQSSGLYSLSSVVTVPSSSLGTQTYICNVNHKPSN |
| | | TKVDKRVEPKSCDKTHTCPPCPAPELLGGPSVFLFP |
| | | PKPKDTLMISRTPEVTCVVVDVSHEDPEVKFNWYV |
| | | DGVEVHNAKTKPREEQYNSTYRVVSVLTVLHQDW |
| | | LNGKEYKCKVSNKALPAPIEKTISKAKGQPREPQVY |
| | | TLPPSREEMTKNQVSLTCLVKGFYPSDIAVEWESNG |
| | | QPENNYKTTPPVLDSDGSFFLYSKLTVDKSRWQQG |
| | | NVFSCSVMHEALHNHYTQKSLSLSPGK |
| 10 | Light Chain | EIVLTQSPDFQSVTPKEKVTITCRASESVDTFGISFM |
| | | NWFQQKPDQSPKLLIHEASNQGSGVPSRFSGSGSGT |
| | | DFTLTINSLEAEDAATYYCQQSKEVPFTFGGGTKVE |
| | | IKRTVAAPSVFIFPPSDEQLKSGTASVVCLLNNFYPR |
| | | EAKVQWKVDNALQSGNSQESVTEQDSKDSTYSLSS |
| | | TLTLSKADYEKHKVYACEVTHQGLSSPVTKSFNRG |
| | | EC |

Inebilizumab and methods of making thereof are described in International PCT Patent Application PCT/US2007/077916, published as WO 2008/031056, which is hereby incorporated by reference (PCT/US2007/077916 refers to VIB551 as “16C4”). Humanized antibodies described herein can be produced using a variety of techniques known in the art, including, but not limited to, CDR-grafting (see e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology 28(4/5):489-498; Studnicka et al., 1994, Protein Engineering, 7(6):805-814; and Roguska et al., 1994, Proc. Natl. Acad Sci., 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000). Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, FW residues in the FW regions will be substituted with the corresponding residue from the CDR donor antibody to alter, preferably improve, antigen binding. These FW substitutions are identified by methods well known in the art, e.g., by modeling of the interactions of the CDR and FW residues to identify FW residues important for antigen binding and sequence comparison to identify unusual FW residues at particular positions. (See, e.g., Queen et al., U.S. Pat. No. 5,585,089; and Riechmann et al., 1988, Nature, 332:323, which are incorporated herein by reference in their entireties.)

In contrast to the anti-CD20 mAb rituximab, Inebilizumab does not mediate complement-dependent cytotoxicity but eliminates B cells via antibody-dependent cellular cytotoxicity and antibody-medicated cellular phagocytosis mechanisms (Herbst et al, 2010).

In embodiments, Inebilizumab is a clear to slightly opalescent, colorless to slightly yellow solution, free from or practically free from visible particles. Inebilizumab is a sterile liquid drug product (100 mg Inebilizumab per vial, nominal) intended for IV infusion following dilution in normal saline. In embodiments, Inebilizumab for IV administration can be supplied as a sterile liquid in a 10R glass vial at a nominal fill volume of 10 mL with 20 mm stopper and flip-off cap overseal. In embodiments, a formulation may be preservative-free. In embodiments. Inebilizumab for IV administration is supplied as a sterile liquid filled at a nominal volume of 10 mL in 10R vials. In embodiments, a vial contains Inebilizumab formulated at 10 mg/mL, in 20 mM histidine/histidine hydrochloride, 70 mM NaCl, 106 mM (4% [w/v]) trehalose dihydrate, and 0.01% (w/v) polysorbate 80, pH 6.0. Alternate formulations are also contemplated and further described in embodiments below.

In embodiments, Inebilizumab can be administered in any form. In embodiments, Inebilizumab is administered intravenously, subcutaneously, orally, intramuscularly, intrathecally, sublingually, rectally, vaginally, cutaneously, systemically, topically, transdermally, or by way of inhalation. In embodiments, Inebilizumab is administered intravenously.

Inebilizumab can be administered at any dose. In embodiments, Inebilizumab is administered at a dose from about 0.5 mg-1000 mg. In embodiments, Inebilizumab is administered at a dose from about, 10 mg-30 mg, 20 mg-100 mg, 50 mg-200 mg, 100 mg-300 mg, 150 mg-350 mg, 200 mg-400 mg, 250 mg-450 mg, 300 mg-500 mg, 300 mg-700 mg, or 400 mg-1000 mg. In embodiments, Inebilizumab is administered at a dose from about 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, or up to about 1000 mg. In embodiments, a subject in need thereof is administered 300 mg.

In embodiments, Inebilizumab may be administered at a dose of about 300 mg. In embodiments, the Inebilizumab may be administered at a dose of about 250 mg to about 350 mg, about 275 mg to about 325 mg, about 290 mg to about 310 mg, about 205 mg to about 305 mg, or it may be a dose of 300 mg. In embodiments, the subject may receive one or more initial doses of Inebilizumab. In embodiments, the subject may receive, one, two, three or more initial doses. In embodiments, the initial dose may be about 300 mg. In embodiments, the Inebilizumab may be administered at an initial dose of about 250 mg to about 350 mg, about 275 mg to about 325 mg, about 290 mg to about 310 mg, about 205 mg to about 305 mg, or an initial dose of 300 mg. In embodiments. Inebilizumab may be administered intravenously with a first initial dose of about 300 mg, a second initial dose of about 300 mg two weeks after the first initial dose, and subsequent doses of about 300 mg every 6 months following the first initial dose.

In embodiments, Inebilizumab is administered with one or more additional therapies. In embodiments, the one or more additional therapies are one or more standard of care therapies. In embodiments, the additional therapy is a corticosteroid. In embodiments, the corticosteroid is prednisone. In embodiments, the one or more additional therapies are one or more of azathioprine, mycophenolate mofetil, mycophenolic acid, or tacrolimus.

In embodiments, Inebilizumab is administered by the minute, hourly, daily, weekly, monthly, or yearly. In embodiments, Inebilizumab is administered twice daily, biweekly, bimonthly, or semiannually. Any number of administrations may be provided to a subject in need thereof. In embodiments, a subject receives from 0-2, 0-3, 0-5, 0-10, 1-3, 1-5, or 1-10 administrations. In embodiments, a subject receives an administration on Day 1, Day 15, and week 26 post treatment initiation. In embodiments, a subject in need thereof is administered 300 mg of Inebilizumab on day 1, day 15, and week 26 post treatment initiation. In embodiments, a subject in need thereof is administered 300 mg of Inebilizumab on day 1, day 15, and week 26 post treatment initiation, and then every 6 months thereafter as needed.

The dose of Inebilizumab that may be used in a method of treating a patient in need thereof, may be a dose administered intravenously at an interval of approximately once every 6 months, or once every 7 months, or once every 8 months, or once every 9 months, or once every 10 months, or once every 11 months, or once a year. In embodiments, Inebilizumab administered in the methods disclosed herein may be at an interval of approximately every 6 months. The approximately every 6 months may be administration every 6 months, every 180 days, every between 170 and 190 days, every between 175 and 185 days, every between 175 and 190 days, or every between 170 and 185 days. The approximately every 6 months may be administration every 26 weeks, every 25 weeks, every 27 weeks, every between 25 and 27 weeks, every between 25 and 26 weeks, or every between 26 and 27 weeks. Prior to the administering the Inebilizumab every approximately 6 months in the methods disclosed herein, an initial Inebilizumab dose may be administered to a subject in need. The initial Inebilizumab dose may be administered approximately 2 weeks before the approximately every 6 month Inebilizumab dosing. The administering the initial Inebilizumab dose approximately 2 weeks before the every approximately 6 month Inebilizumab dosing may be the administering of the initial Inebilizumab dose 12 days, 13 days, 14 days, 15 days, or 16 days before the approximately 6 months Inebilizumab dosing. The initial Inebilizumab dose may or may not be co-administered with oral corticosteroids or any standard of treatment dose. In embodiments, Inebilizumab is administered intravenously.

In embodiments, the disclosure is directed to a method of treatment comprising: administering Inebilizumab to a patient in need of treatment for IgG4-RD flare, wherein the Inebilizumab is administered intravenously at a dose of 300 mg every 6 months. In embodiments, two weeks prior to the administering the 300 mg Inebilizumab every 6 months, an initial 300 mg Inebilizumab dose is administered to the subject. In embodiments, the disclosure is directed to a method of treating a patient diagnosed with IgG4-RD and/or IgG4-RD flare, the method comprising: administering Inebilizumab to a patient in need of treatment, wherein the Inebilizumab is administered intravenously with a first initial dose of 300 mg, a second initial dose of 300 mg two weeks after the first initial dose, and subsequent doses of 300 mg every 6 months following the first initial dose.

In embodiments, an anti-CD19 binding agent that is not Inebilizumab may be utilized to treat or prevent IgG4-RD and/or IgG4-RD flare. Exemplary anti-CD19 binding agents that may be administered comprise: XmAb5871, AFM11, MOR-208, Blinatumomab, SAR3419, SGN-19A, MDX-1342, Lonvastuximab, and combinations thereof. In embodiments, another anti-CD19 antibody may be substituted in any of the methods disclosed herein. In embodiments, the other anti-CD19 antibody, if other than Inebilizumab, may be any of, for example, MOR00208 (also referred to as Xmab 5574 or tafasitamab; disclosed in U.S. Patent Application No. 20170137516, which is incorporated herein by reference in its entirety); blinatumomab (Amgen; Astellas; MicroMet); loncastuximab tesirine (ADC Therapeutics); GTB-1550/OXS-1550 (Oxis Biotech Inc); obexelimab/XmAb5871 (Xencor Inc); AFM11 (Affimed); or coltuximab/ravtansine (ImmunoGen Inc).

In embodiments, polynucleotides comprising a nucleotide sequence encoding a human, humanized, or chimeric anti-CD19 antibody of the disclosure are provided. The disclosure also encompasses polynucleotides that hybridize under stringent or lower stringency hybridization conditions, as defined herein, to polynucleotides that encode a human, humanized, or chimeric antibody that specifically binds to human CD19. In embodiments, provided is a vector comprising one or more nucleotide sequences encoding a human, humanized, or chimeric anti-CD19 antibody described herein. The present disclosure further relates to an isolated cell comprising a vector wherein said vector comprises one or more nucleotide sequences encoding a human, humanized, or chimeric anti-CD19 antibody of the disclosure. In embodiments, chimeric, human, and humanized anti-CD19 monoclonal antibodies described herein include those of the IgG1, IgG2, IgG3, or IgG4 human isotype.

In embodiments, a humanized anti-CD19 antibody described herein mediates antibody-dependent cellular cytotoxicity (ADCC), complement-dependent cell-mediated cytotoxicity (CDC), and/or apoptosis. In embodiments, a humanized anti-CD19 antibody described herein inhibits anti-IgM/CpG stimulated B cell proliferation.

### Additional Therapeutics

In embodiments, Inebilizumab may be administered with one or more additional therapeutics. In embodiments, the one or more additional therapeutics are administered as part of a preparative regimen. In embodiments, Inebilizumab is co-administered with one or more additional therapeutics. In embodiments, Inebilizumab is administered after one or more additional therapeutics. In embodiments, one or more additional therapeutics follow administration of Inebilizumab. In embodiments, an additional therapeutic is administered for prophylaxis of infusion reactions.

### Corticosteroid

In embodiments, an additional therapeutic comprises a corticosteroid. In embodiments, a corticosteroid comprises a glucocorticoid. Glucocorticoids (GCs) are steroid hormones used for the treatment of inflammation, autoimmune diseases, and/or cancer. To exert their broad physiological and therapeutic effects, GCs bind to the GC receptor (GR) which belongs to the nuclear receptor superfamily of transcription factors. In embodiments, one or more GCs are administered to a subject in need thereof. In embodiments, one or more GCs is selected from the group consisting of: triamcinolone, methylprednisolone, triamcinolone, budesonide, dexamethasone, triamcinolone, prednisone, hydrocortisone, dexamethasone, betamethasone, prednisolone, deflazacort, and combinations thereof. In embodiments, a GCs is prednisone.

In embodiments, a glucocorticoid such as prednisone, or an equivalent, is administered upon detection of an IgG4-RD flare. In embodiments, a subject in need thereof has previously been administered a corticosteroid, such as a glucocorticoid. In embodiments, a corticosteroid, such as a glucocorticoid is administered to a subject in need thereof. In some embodiments, post Inebilizumab administration, the corticosteroid treatment can be tapered off. In embodiments, prednisone, or another suitable GC provided herein, is tapered off over a period of days, or weeks. In embodiments, tapering occurs over a period of weeks. In embodiments, tapering of prednisone or another suitable GC occurs over a period of about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks. In embodiments, the tapering of the GC treatment overlaps with administration of Inebilizumab. In embodiments, the overlapping period comprises at least about 0-5 days, 1-10 days, 1-14 days, 1-20 days, or 1-30 days. In embodiments, the overlapping period comprises at least about or at most about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or up to about 50 days. In embodiments, during a tapering period, a subject is administered a GC at a dosage over a period of 1 week, 2 weeks, or 3 weeks. In embodiments, a subject is administered prednisone at decreasing dosages, each lasting 2 weeks. In embodiments, a subject is administered prednisone at dosages of about 20 mg/day, 15 mg/day, and 5 mg/day, each lasting 2 weeks.

In embodiments, an administration of a GC comprises at least about or at most about: 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, 20 mg/day, 21 mg/day, 22 mg/day, 23 mg/day, 24 mg/day, 25 mg/day, 26 mg/day, 27 mg/day, 28 mg/day, 29 mg/day, 30 mg/day, 31 mg/day, 32 mg/day, 33 mg/day, 34 mg/day, 35 mg/day, 36 mg/day, 37 mg/day, 38 mg/day, 39 mg/day, 40 mg/day, 41 mg/day, 42 mg/day, 43 mg/day, 44 mg/day, 45 mg/day, 46 mg/day, 47 mg/day, 48 mg/day, 49 mg/day, or 50 mg/day. In embodiments, a subject is administered methylprednisolone at a dosage of 100 mg/day.

### Antipyretic

In embodiments, an additional therapeutic comprises an antipyretic. In embodiments, an antipyretic is administered as a prophylaxis. In embodiments, an antipyretic is an NSAID, paracetamol, or metamizole. In embodiments, the antipyretic is an NSAID selected from the group consisting of ibuprofen, naproxen, ketoprofen, and nimesulide, aspirin, and related salicylates such as choline salicylate, magnesium salicylate, sodium salicylate, phenazone, and combinations thereof. In embodiments, an antipyretic is paracetamol. In embodiments, an antipyretic is metamizole.

In embodiments, an antipyretic is administered at any time period during a treatment regimen. In embodiments, an antipyretic is administered prior to, during, or after an administration of Inebilizumab. In embodiments, an antipyretic is administered from about 30-60 minutes ahead of an administration of Inebilizumab.

In embodiments, the administration of an antipyretic comprises at least about or at most about: 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, 600 mg, 610 mg, 620 mg, 630 mg, 640 mg, 650 mg, 660 mg, 670 mg, 680 mg, 690 mg, 700 mg, 710 mg, 720 mg, 730 mg, 740 mg, 750 mg, 760 mg, 770 mg, 780 mg, 790 mg, 800 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg, or up to about 1000 mgs. In embodiments, a subject is administered 500-650 mg or equivalent of an antipyretic.

### Antihistamine

In embodiments, an additional therapeutic comprises an antihistamine. In embodiments, an antihistamine is selected from the group consisting of: acrivastine, azatadine, brompheniramine, chlophiheniramine, clemastine, cyproheptadine, dexbromphenir amine, dimenhydrinate, diphenhydramine, doxylamine, hydroxyzine, meclizine, phenindamine, phenyltoloxamine, promethazine, pyrilamine, tripelennamine, triprolidine, and combinations thereof. In embodiments, a non-sedating antihistamine is utilized and can include, but is not limited to, astemizole, cetirizine, ebastine, fexofenadine, loratidine, terfenadine, and combinations thereof. In embodiments, the antihistamine is diphenhydramine.

In embodiments, an antihistamine is administered at any time period during a treatment regimen. In embodiments, an antihistamine is administered prior to, during, or after an administration of Inebilizumab. In embodiments, an antihistamine is administered from about 30-60 minutes ahead of an administration of Inebilizumab.

In embodiments, an administration of an antihistamine comprises at least about or about most about: 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, 20 mg/day, 21 mg/day, 22 mg/day, 23 mg/day, 24 mg/day, 25 mg/day, 26 mg/day, 27 mg/day, 28 mg/day, 29 mg/day, 30 mg/day, 31 mg/day, 32 mg/day, 33 mg/day, 34 mg/day, 35 mg/day, 36 mg/day, 37 mg/day, 38 mg/day, 39 mg/day, 40 mg/day, 41 mg/day, 42 mg/day, 43 mg/day, 44 mg/day, 45 mg/day, 46 mg/day, 47 mg/day, 48 mg/day, 49 mg/day, or 50 mg/day. In embodiments, a subject is administered diphenhydramine at a dosage of about 25-50 mg.

In embodiments, administration of Inebilizumab to a subject in need thereof is effective in preventing new administration of one or more additional therapeutics provided herein. In embodiments, administration of Inebilizumab to a subject in need thereof is effective in preventing new administration of a corticosteroid provided herein. In embodiments, administration of Inebilizumab to a subject in need thereof is effective in preventing new administration of an antihistamine provided herein. In embodiments, administration of Inebilizumab to a subject in need thereof is effective in preventing new administration of an antipyretic provided herein. In embodiments, administration of Inebilizumab to a subject in need thereof is effective in preventing increased administration of a corticosteroid provided herein. In embodiments, administration of Inebilizumab to a subject in need thereof is effective in preventing increased administration of an antihistamine provided herein. In embodiments, administration of Inebilizumab to a subject in need thereof is effective in preventing increased administration of an antipyretic provided herein.

### Steroid-Sparing Agent

In embodiments, an additional therapeutic comprises a steroid-sparing agent. A steroid-sparing agent may be utilized to reduce use of steroids. In embodiments, a steroid-sparing agent is utilized to reduce or eliminate steroid administration. In embodiments, a steroid-sparing agent is utilized to reduce or maintain a low steroid dose. Steroid-sparing agents are selected from the group consisting of azathioprine, mycophenolate mofetil, cyclosporine, methotrexate, leflunomide, cyclophosphamide, rituximab, etanercept, adalimumab, infliximab, certolizumab pegol, golimumab, abatacept, and tocilizumab. In embodiments, a subject is not administered a steroid-sparing agent. In embodiments, administration of Inebilizumab is effective in reducing or eliminating administration of a steroid-sparing agent in a subject in need thereof. In embodiments, administration of Inebilizumab is effective in reducing or eliminating administration of a steroid-sparing agent by about 1-fold, 5-fold, 10-fold, 15-fold, 20-fold, 25-fold, 30-fold, 35-fold, 40-fold, 45-fold, or 50-fold as compared to an otherwise comparable subject lacking the administration of Inebilizumab.

### Secondary Immunotherapy.

In embodiments, administration of a secondary immunotherapy is provided herein. In embodiments, a secondary immunotherapy comprises antibody therapy. In embodiments, an antibody therapy comprises an antibody that binds a B-cell. An antibody therapy can comprise an anti-CD19 binding agent and/or an anti-CD20 binding agent. B-cell binding antibodies are selected from the group consisting of rituximab, ocrelizumab, ofatumumab, obinutuzumab, ibritumomab, and combinations thereof. In embodiments, a secondary immunotherapy is selected from the group consisting of anti-CD20 mAb, anti-CD52 mAb, anti-CD22 antibody, and anti-CD20 antibodies, such as RITUXAN™ (C2B8; RITUXIMAB™; IDEC Pharmaceuticals). Other examples of secondary immunotherapies that can be used in combination with antibodies of the disclosure or used in compositions of the disclosure include, but are not limited to, HERCEPTIN™ (Trastuzumab; Genentech), MYLOTARG™ (Gemtuzumab ozogamicin; Wyeth Pharmaceuticals), CAMPATH™ (Alemtuzumab; Berlex), ZEVALIN™ (Ipritumomab tiuxetan; Biogen Idec), BEXXAR™ (Tositumomab; GlaxoSmithKline Corixa), ERBITUX™ (Cetuximab; Imclone), and AVASTIN™ (Bevacizumab; Genentech).

In embodiments, a secondary immunotherapy comprises an antibody specific for an Fc receptor selected from the group consisting of FcγRI, FcγRIIA, FcγRIIB, FcγRIII and/or FcγRIV. In embodiments, a secondary immunotherapy comprises an antibody specific for FcγRIIB Anti-FcγRIIB antibodies suitable for this purpose have been described in US Patent Application Publication No. 2004185045 (U.S. Pat. No. 7,425,620), PCT Publication Nos. WO05051999A, WO05018669 and WO04016750.

In embodiments, an anti-CD19 binding agent and an anti-CD20 and/or an anti-CD22 mAb and/or an anti-CD52 mAb can be administered, optionally in the same pharmaceutical composition, in any suitable ratio. To illustrate, the ratio of the anti-CD19 and anti-CD20 antibody can be a ratio of about 1000:1, 500:1, 250:1, 100:1, 90:1, 80:1, 70:1, 60:1, 50:1, 40:1, 30:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50, 1:60, 1:70, 1:80, 1:90, 1:100, 1:250, 1:500 or 1:1000 or more. Likewise, the ratio of the anti-CD19 and anti-CD22 antibody can be a ratio of about 1000:1, 500:1, 250:1, 100:1, 90:1, 80:1, 70:1, 60:1, 50:1, 40:1, 30:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50, 1:60, 1:70, 1:80, 1:90, 1:100, 1:250, 1:500 or 1:1000 or more. Similarly, the ratio of the anti-CD19 and anti-CD52 antibody can be a ratio of about 1000:1, 500:1, 250:1, 100:1, 90:1, 80:1, 70:1, 60:1, 50:1, 40:1, 30:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50, 1:60, 1:70, 1:80, 1:90, 1:100, 1:250, 1:500 or 1:1000 or more.

### Methods

In embodiments, provided herein are methods of using provided compositions and pharmaceutical compositions for the treatment of IgG4-RD. In embodiments, provided herein are methods of using provided compositions and pharmaceutical compositions for the treatment or prevention of an IgG4-RD flare. In embodiments, provided are methods of treating IgG4-RD, comprising administering to a subject in need thereof an effective amount of Inebilizumab, wherein the administering is sufficient to reduce or eliminate an IgG4-RD flare, thereby treating the IgG4-RD. In embodiments, provided are methods of treating IgG4-RD, comprising administering to a subject in need thereof an effective amount of Inebilizumab, wherein the administering is sufficient to extend time to detection of a IgG4-RD flare by at least 1-fold as compared to an otherwise comparable subject lacking the administering. Also provided are methods of treating IgG4-RD comprising administering to a subject in need thereof an effective amount of an anti-CD19 binding agent, wherein the administering is sufficient to extend time to detection of a IgG4-RD flare by at least 1-fold as compared to an otherwise comparable subject lacking the administering.

In embodiments, compositions of the disclosure are administered to a human subject as long as the subject is responsive to therapy. In embodiments, Inebilizumab is administered to a human subject as long as the subject's disease does not progress. In embodiments, Inebilizumab is administered to a human subject until a subject's disease does not progress or has not progressed for a period of time, then the subject is not administered Inebilizumab unless the disease reoccurs or begins to progress again. For example, a subject can be treated with any of the above doses for about 4 to 8 weeks, during which time the subject is monitored for disease progression. If disease progression stops or reverses, then the subject will not be administered Inebilizumab until that subject relapses, i.e., the disease being treated reoccurs or progresses. Upon this reoccurrence or progression, the subject can be treated again with the same dosing regimen initially used or using other doses described herein.

In embodiments, Inebilizumab can be administered as a loading dose followed by multiple lower doses (maintenance doses) over a period of time. In embodiments, the doses may be timed, and the amount adjusted to maintain effective B cell reduction or elimination. In embodiments, the loading dose is about 10, 11, 12, 13, 14, 15, 16, 17, or 18 mg/kg of subject body weight and the maintenance dose is at least about 5 to 10 mg/kg of subject body weight. In other embodiments, the maintenance dose is administered at intervals of every 7, 10, 14 or 21 days. In embodiments. Inebilizumab can be administered absent a loading dose.

In embodiments, administration of Inebilizumab may enable the postponement of toxic therapy and may help avoid unnecessary side effects and the risks of complications associated with glucocorticoids. In embodiments, toxic therapies are delayed in subjects administered Inebilizumab for up to about 6 months, 1, 2, 3, 4, 5, 6, 7, 8, 9, or 10 years.

In embodiments, Inebilizumab is administered as a single agent therapy or monotherapy. In embodiments, Inebilizumab is administered in a combination therapy. In embodiments, post-administration a subject may be assessed for any one of the below referenced assessments.

### Immunogenicity Assessment

In embodiments, a method provided herein can comprise determining immunogenicity against an anti-CD19 binding agent, such as Inebilizumab, in a subject in need thereof post administration. In embodiments, immunogenicity comprises determining a level of anti-drug antibodies (ADA). In embodiments, a suitable assay for detecting ADA comprises an electrochemiluminescence (ECL) assay, a bead-based assay, a cell-based assay, and combinations thereof. In embodiments, ADA against Inebilizumab is not detected. In embodiments, ADA against Inebilizumab is at most about 1-fold, 5-fold, 10-fold, 15-fold, 20-fold, 25-fold, 30-fold, 35-fold, 40-fold, 45-fold, or 50-fold as compared to an otherwise comparable anti-CD19 binding agent; or as compared to an otherwise comparable subject lacking the administering of Inebilizumab.

### Pharmacodynamic Assessment

In embodiments, a method provided herein can comprise determining an amount of reduction or elimination of CD19 positive cells by an anti-CD19 binding agent, such as Inebilizumab, in a subject in need thereof post administration. In embodiments, an assessment can occur over a period of time. In embodiments, a reduction of CD19 positive cells is detected as compared to an otherwise comparable method lacking the administering of Inebilizumab or an anti-CD19 binding agent. In embodiments, elimination of CD19 positive cells is detected as compared to an otherwise comparable method lacking the administering of Inebilizumab or an anti-CD19 binding agent. In embodiments, peripheral B-cell and plasma cell biomarkers such as, absolute counts and corresponding percentage based on baseline, are obtained. In embodiments, reduction of anti-CD19 positive cells comprises at least about or at most about: 1-fold, 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 105-fold, 110-fold, 115-fold, 120-fold, 125-fold, 130-fold, 135-fold, 140-fold, 145-fold, 150-fold, or up to about 200-fold reduction of CD19 positive cells as compared to an otherwise comparable method lacking the administering.

The disclosure provides for antibodies, such as Inebilizumab, that efficiently reduce or deplete B cells in a human subject. Because Inebilizumab binds to and depletes CD19+ cells, the reduction or elimination can be used as a measure of treatment effect. In embodiments, treatment efficacy of Inebilizumab or an anti-CD19 binding agent on peripheral B cell counts can be assessed over time using a suitable immunoassay. Suitable immunoassays comprise flow cytometry, histology, immunohistochemistry, blood analysis, microscopy, PCR, ELISA, and combinations thereof. In embodiments, an immunoassay comprises obtaining a blood sample from a subject in need thereof and performing anti-CD19 cellular staining on cells. Inebilizumab may also be used in a method of treating a subject in need thereof in which the Inebilizumab is administered at a dose that (i) depletes at least 900% of circulating CD20+ B cells for at least six months, and (ii) does not increase risk of infections in the subject. The dose that depletes the at least 90% of circulating CD20+ B cells for at least six months may also deplete peripheral blood CD20− plasmablasts and plasma cells. The dose that depletes the at least 90% of circulating CD20+ B cells may deplete the circulating CD20+ B cells for longer than six months. It may deplete the at least 90% of circulating CD20+ B cells for at least 9 months or at least 1 year.

Methods for measuring depletion of B cells that can be used in connection with compositions and methods of the disclosure are well known in the art and include but are not limited to the following embodiments. In embodiments, circulating B cell depletion can be measured with flow cytometry using a reagent other than an anti-CD19 antibody that binds to B cells to define the amount of B cells. In embodiments, B cell levels in the blood can be monitored using standard serum analysis. In embodiments, B cell depletion is indirectly measured by defining the amount to an antibody known to be produced by B cells. The level of that antibody is then monitored to determine the depletion and/or functional depletion of B cells. In embodiments, B cell depletion can be measured by immunochemical staining to identify B cells. In embodiments, B cells or tissues or serum comprising B cells extracted from a patient can be placed on microscope slides, labeled and examined for presence or absence. In related embodiments, a comparison is made between B cells extracted prior to therapy and after therapy to determine differences in the presence of B cells.

Inebilizumab may be used in a method of treating a patient in need of treatment for IgG4-RD, in which the Inebilizumab is administered at a dose that depletes at least about 20% of circulating CD20+ B cells for at least 6 months. In embodiments, Inebilizumab may achieve at least about 20%, at least about 30%, at least about 40%, at least about 50%, at least about 60%, at least about 70%, at least about 80%, at least about 90%, at least about 95%, or about 100% B cell depletion. In embodiments, Inebilizumab is administered at a dose that depletes at least about 90% of circulating CD20+ B cells for at least 6 months. In embodiments, Inebilizumab may deplete B cell subsets in a human subject. In embodiments, Inebilizumab may deplete circulating B cells, blood B cells, splenic B cells, marginal zone B cells, follicular B cells, peritoneal B cells, and/or bone marrow B cells. CD19 is present on the surface of B cells at all developmental stages. Inebilizumab may therefore deplete B cells of all developmental stages. In embodiments, Inebilizumab may achieve depletion of progenitor B cells, early pro-B cells, late pro-B cells, large-pre-B cells, small pre-B cells, immature B cells, mature B cells, antigen stimulated B cells, and/or plasma cells.

Reduction or elimination of B cells may persist for extended periods of time. In embodiments, B cell depletion may persist for at least 1 day, at least 2 days, at least 3 days, at least 4 days, at least 5 days at least 6 days, at least 7 days, at least 8 days, at least 9 days, at least 10 days, at least 15 days, at least 20 days, at least 25 days, or at least 30 days. In another embodiment, B cell depletion by Inebilizumab may persist for at least 1 week, at least 2 weeks, at least 3 weeks, at least 4 weeks, at least 5 weeks, at least 6 weeks, at least 7 weeks, at least 8 weeks, at least 9 weeks, or at least 10 weeks. In embodiments, B cell depletion may persist for at least 1 month, at least 2 months, at least 3 months, at least 4 months, at least 5 months, at least 6 months, at least 7 months, at least 8 months, at least 9 months, at least 10 months, at least 11 months or at least 12 months.

In embodiments, Inebilizumab inhibits in vitro stimulated B cell proliferation. In embodiments. Inebilizumab inhibits in vitro B cell proliferation induced by anti-IgM/CpG or anti-IgM/CD40 stimulation. In embodiments, Inebilizumab inhibits in vitro stimulated B cell proliferation by at least about 10%, at least about 20%, at least about 30%, at least about 40%, at least about 50% or at least about 75%.

### Pharmacokinetic Assessment

In embodiments, a method provided herein can comprise determining a concentration of an anti-CD19 binding agent, such as Inebilizumab, in a subject in need thereof post administration. In embodiments, a sample is a blood sample or serum sample. Inebilizumab is degraded by proteolytic enzymes widely distributed in the body. In embodiments, the estimated Inebilizumab systemic clearance (CL) of the first-order elimination pathway can be about 0.19 L/day. In embodiments, a suitable assay to measure pharmacokinetics comprises electrochemiluminescence (ECL) assay, a bead-based assay, a cell-based assay, and combinations thereof. In embodiments, a sample comprises serum and the serum is accessed for Inebilizumab serum concentration by measuring: maximum observed concentration (Cₘₐₓ), area under the concentration-time curve (AUC), CL, and terminal elimination half-life (t_{1/2}).

### Additional Assessments

Subjects administered any of the compositions provided herein may undergo assessments post administration. In embodiments, an assessment can determine an effect of Inebilizumab on measures of disease activity. For example, a subject may undergo an assessment to determine subject functioning as measured by changes from baseline to post treatment in SF-36v2, FACIT-fatigue, patient global assessment of disease activity of combinations thereof. In embodiments, an annualized flare rate may also be calculated.

In embodiments, an additional assessment explores health resource utilization in subject with IgG4-RD. Suitable metrics comprise determining the number of inpatient hospitalizations, days of hospitalization, days in an intensive care unit, emergency department visits, non-study related physician visits, home-health visits (physician or nurse), disease-related imaging procedures, and disease-related procedures/surgeries (stenting, other).

In embodiments, an additional assessment determines effects of Inebilizumab administration on circulating immunoglobulins. For example, a subject may be assessed to determine a change from baseline in immunoglobulin levels (IgG, IgG subclasses including IgG4, IgM, IgA, IgE, and total immunoglobulins).

In embodiments, an additional assessment evaluates effects of Inebilizumab on complement components and/or the ELF score by way of evaluating changes from baseline in serum levels of complement components C3 and C4 and the Enhanced Liver Fibrosis (ELF) score. An ELF score is an ECM marker set consisting of tissue inhibitor of metalloproteinases 1 (TIMP-1), amino-terminal propeptide of type III procollagen (PIIINP) and hyaluronic acid (HA) showing good correlations with fibrosis stages in chronic liver disease.

In embodiments, an additional assessment evaluates effects of Inebilizumab on other disease-relevant measures, including other immune cell populations, biomarkers, and gene expression in subjects with IgG4 RD.

In embodiments, an additional assessment evaluates the relationship, if any, between genetic variations, disease activity, and efficacy by way of changes from baseline in (a) blood gene expression profiles: (b) serum biomarker expression (e.g. inflammation-related cytokines/chemokines); or (c) (a) and (b).

In embodiments, an additional assessment comprises completing the IgG4-RD Responder Index (RI). The RI can be used as an assessment tool of disease activity and damage. The RI evaluates disease activity and damage in 25 domains, incorporating higher weights for disease manifestations that require treatment urgently or that worsen despite treatment, see Wallace Z S, Khosroshahi A, Carruthers M D, Perugino C A, Choi H, Campochiaro C, et al. An international multispecialty validation study of the IgG4-related disease responder index. Arthritis Care Res (Hoboken). 2018; 70(11):1671-78 incorporated herein by reference in its entirety. In embodiments, the RI evaluates diseases activity at any disease site selected from the group consisting of: meninges, orbital lesion, parotid gland, other salivary gland, nasal cavity lesion, other ENT lesion, lung, aorta/large blood vessel, retroperitoneal fibrosis, sclerosing mesenteritis, liver, kidney, constitutional symptoms (weight loss, fever, fatigue due to IgG4-RD, pituitary gland, lacrimal gland, submandibular gland, mastoiditis/middle ear disease, sinusitis, thyroid, lymph node, heart/pericardium, sclerosing mediastinitis, pancreas, bile duct, skin, other, and any combinations thereof. In embodiments, disease activity at each of the aforementioned cites is scored on a scale of 0 to 4, where 0=unaffected or resolved; 1=improved but persistent: 2=new or recurrence (while off of treatment) or unchanged; and 3=worse or new (despite treatment). In embodiments, complete remission is defined as an IgG4-RD Responder Index score of 0 at Week 52 post treatment, no flare, and no treatment for flare or disease control except for glucocorticoid taper if needed. In embodiments, a subject administered any of the compositions provided herein has a responder index of: 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75. In embodiments, a subject administered any of the compositions provided herein has a responder index measured at a point, post administration, of: 0 days, 5 days, 10 days, 15 days, 20 days, 25 days, 30 days, 35 days, 40 days, 45 days, 50 days, 55 days, 60 days, 65 days, 70 days, 75 days, 80 days, 85 days, 90 days, 95 days, 100 days, 105 days, 110 days, 115 days, 120 days, 125 days, 130 days, 135 days, 140 days, 145 days, 150 days, 155 days, 160 days, 165 days, 170 days, 175 days, 180 days, 185 days, 190 days, 195 days, 200 days, 205 days, 210 days, 215 days, 220 days, 225 days, 230 days, 235 days, 240 days, 245 days, 250 days, 255 days, 260 days, 265 days, 270 days, 275 days, 280 days, 285 days, 290 days, 295 days, 300 days, 305 days, 310 days, 315 days, 320 days, 325 days, 330 days, 335 days, 340 days, 345 days, 350 days, 355 days, 360 days, or 365 days. In embodiments, the RI is 0 at 10 days, 30 days, 50 days, 100 days, 150 days, 200 days, 250 days, 300 days, or 375 days post administration. In embodiments, the RI is 0-10, 5-20, 10-30, 15-40, 20-50, or 25-60 at 10 days, 30 days, 50 days, 100 days, 150 days, 200 days, 250 days, 300 days, or 375 days post administration. In embodiments, the RI is 0-10, 5-20, 10-30, 15-40, 20-50, or 25-60 at 10-50 days, 20-100 days, 30-150 days, 40-200 days, 50-100 days, 50-200 days, 100-200 days, 150-250 days, 200-300 days, or 300-375 days post administration. In embodiments, the RI can be reduced by at least about: 5%, 10%, 15%, 20%, 25%, 30%, 35%, 40%, 45%, 50%, 55%, 60%, 65%, 70%, 75%, 80%, 85% 90%, 95%, or 10% post administration. In embodiments, the RI can be reduced by at least about: 5%, 10%, 15%, 20%, 25%, 30%, 35%, 40%, 45%, 50%, 55%, 60%, 65%, 70%, 75%, 80%, 85%, 90%, 95%, or 100% by day: 5, 10, 15, 20, 25, 30, 35, 40, 45, 50, 55, 60, 65, 70, 75, 80, 85, 90, 95, 100, 105, 110, 115, 120, 125, 130,135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, or day 375 post administration.

Any of the aforementioned additional assessments may determine that administration of Inebilizumab or an anti-CD19 binding agent provided herein does not result in a change from baseline of an evaluated metric (e.g. such as any of the metrics described herein) in a subject in need thereof. In embodiments, administration of Inebilizumab or an anti-CD19 binding agent provided herein results in an increase in an evaluated metric in a subject in need thereof as compared to a baseline level of that metric. In embodiments, administration of Inebilizumab or an anti-CD19 binding agent provided herein results in a decrease in an evaluated metric in a subject in need thereof as compared to a baseline level of that metric. Suitable metrics are those described herein that include but are not limited to: immunogenicity, pharmacokinetic, pharmacodynamic, disease activity, subject functionality, immunoglobulin levels, ELF score, complement components, genetic variations, disease-relevant measures, and combinations thereof.

### Pharmaceutical Composition

In embodiments, a pharmaceutical composition is provided herein. In some embodiments, the pharmaceutical composition comprises Inebilizumab. In embodiments a pharmaceutical composition is part of a therapeutic regimen that comprises Inebilizumab, and one or more additional therapeutics provided herein.

Many drugs can be administered orally as liquids, capsules, tablets, or chewable tablets. Because the oral route is the most convenient and usually the safest and least expensive, it is the one most often used. However, it has limitations because of the way a drug typically moves through the digestive tract. For drugs administered orally, absorption may begin in the mouth and stomach. However, most drugs are usually absorbed from the small intestine. The drug passes through the intestinal wall and travels to the liver before being transported via the bloodstream to its target site. The intestinal wall and liver chemically alter (metabolize) many drugs, decreasing the amount of drug reaching the bloodstream. Consequently, these drugs are often given in smaller doses when injected intravenously to produce the same effect.

For a subcutaneous route, a needle is inserted into fatty tissue just beneath the skin. After a drug is injected, it then moves into small blood vessels (capillaries) and is carried away by the bloodstream. Alternatively, a drug reaches the bloodstream through the lymphatic vessels. The intramuscular route is preferred to the subcutaneous route when larger volumes of a drug product are needed. Because the muscles lie below the skin and fatty tissues, a longer needle is used. Drugs are usually injected into the muscle of the upper arm, thigh, or buttock. How quickly the drug is absorbed into the bloodstream depends, in part, on the blood supply to the muscle: The sparser the blood supply, the longer it takes for the drug to be absorbed. For the intravenous route, a needle is inserted directly into a vein. A solution containing the drug may be given in a single dose or by continuous infusion. For infusion, the solution is moved by gravity (from a collapsible plastic bag) or, more commonly, by an infusion pump through thin flexible tubing to a tube (catheter) inserted in a vein, usually in the forearm.

In embodiments, a pharmaceutical composition provided herein is administered via infusion. An infusion can take place over a period of time. For example, an infusion can be an administration of a pharmaceutical over a period of about 5 minutes to about 10 hours. An infusion can take place over a period of about 5 min, 10 min, 20 min, 30 min, 40 min, 50 min, 1 hour, 1.5 hours, 2 hours, 2.5 hours, 3 hours, 3.5 hours, 4 hours, 4.5 hours, 5 hours, 6 hours, 7 hours, 8 hours, 9 hours, or up to about 10 hours. In embodiments, intravenous administration is used to deliver a precise dose quickly and in a well-controlled manner throughout the body. It is also used for irritating solutions, which would cause pain and damage tissues if given by subcutaneous or intramuscular injection. When given intravenously, a drug is delivered immediately to the bloodstream and tends to take effect more quickly than when given by any other route. Consequently, health care practitioners closely monitor people who receive an intravenous injection for signs that the drug is working or is causing undesired side effects. Also, the effect of a drug given by this route tends to last for a shorter time. Therefore, some drugs must be given by continuous infusion to keep their effect constant. In embodiments, infusion reactions can occur and include headache, nausea, somnolence, dyspnea, fever, myalgia, rash, or other symptoms. Potential risks associated with administration of Inebilizumab are infection, redness, swelling, pain, and induration at the administration site. Prior to each IV infusion subjects may receive prophylaxis with IV methylprednisolone, oral diphenhydramine, and oral acetaminophen, or equivalent(s) to reduce the risk or severity of potential reactions.

In embodiments, a pharmaceutical is administered intrathecally. For the intrathecal route, a needle is inserted between two vertebrae in the lower spine and into the space around the spinal cord. The drug is then injected into the spinal canal. A small amount of local anesthetic is often used to numb the injection site. This route is used when a drug is needed to produce rapid or local effects on the brain, spinal cord, or the layers of tissue covering them (meninges)—for example, to treat infections of these structures.

Drugs administered by inhalation through the mouth can be atomized into smaller droplets than those administered by the nasal route, so that the drugs can pass through the windpipe (trachea) and into the lungs. How deeply into the lungs they go depends on the size of the droplets. Smaller droplets go deeper, which increases the amount of drug absorbed. Inside the lungs, they are absorbed into the bloodstream. Drugs applied to the skin are usually used for their local effects and thus are most commonly used to treat superficial skin disorders, such as psoriasis, eczema, skin infections (viral, bacterial, and fungal), itching, and dry skin. The drug is mixed with inactive substances. Depending on the consistency of the inactive substances, the formulation may be an ointment, cream, lotion, solution, powder, or gel

In embodiments, a pharmaceutical composition can be administered either alone or together with a pharmaceutically acceptable carrier or excipient, by any routes, and such administration can be carried out in both single and multiple dosages. More particularly, a pharmaceutical composition can be combined with various pharmaceutically acceptable inert carriers in the form of tablets, capsules, lozenges, troches, hand candies, powders, sprays, aqueous suspensions, injectable solutions, elixirs, syrups, and the like. Such carriers include solid diluents or fillers, sterile aqueous media and various non-toxic organic solvents, etc. Moreover, pharmaceutical formulations can be suitably sweetened and/or flavored by means of various agents of the type commonly employed for such purposes. Exemplary carriers and excipients can include dextrose, sodium chloride (NaCl), sucrose, lactose, cellulose, xylitol, sorbitol, maltitol, gelatin, PEG, PVP, histidine/histidine hydrochloride, trehalose dihydrate, polysorbate 80, and any combination thereof. In embodiments, an excipient comprises: histidine/histidine hydrochloride, NaCl, trehalose dihydrate, and polysorbate 80.

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world, or that they disclose essential matter.

### EXAMPLES

### Example 1: A Phase 3, Randomized, Double-blind, Multicenter, Placebo-Controlled Study of Inebilizumab Efficacy and Safety in IgG4 Related Disease

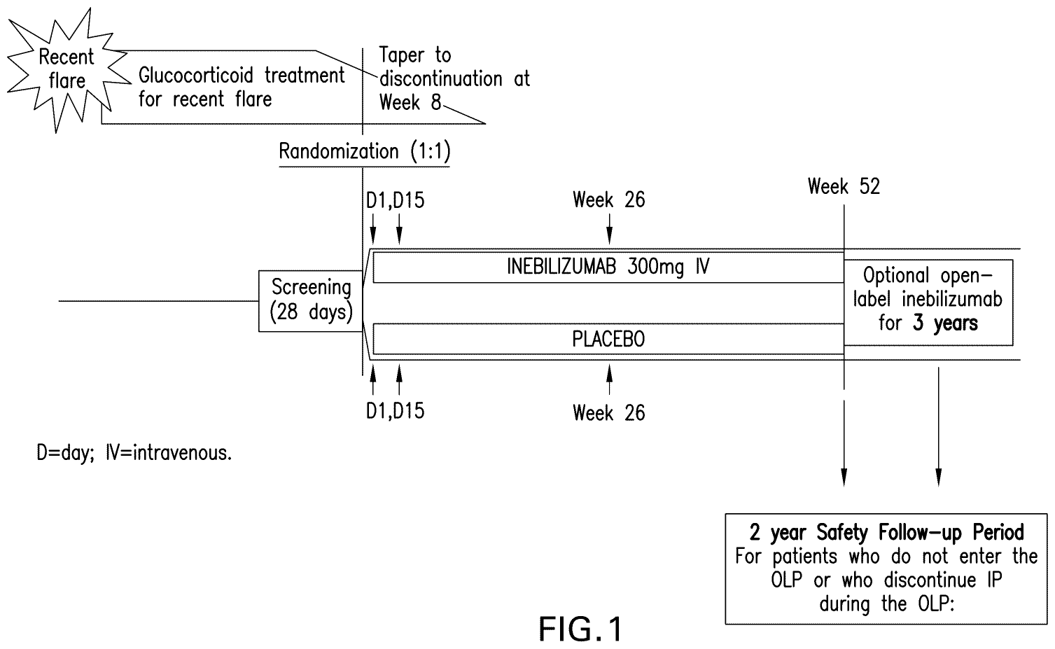

This is a multicenter, randomized, double-blind, placebo-controlled, parallel-cohort study to evaluate the efficacy and safety of Inebilizumab for prevention of disease flare in adults with active IgG4-RD who are at high risk of recurrent flare. FIG. 1 presents the overall study design.

### Population

Adults who have reached the age of consent in the applicable region (e.g., ≥18 years in the US or ≥20 years in Japan) who have recently active IgG4-RD (initial diagnosis or flare) that requires new or ongoing treatment, are at high risk of future flare (as evidenced by multi-organ disease involvement) and meet the 2019 American College of Rheumatology/European League Against Rheumatism (ACR/EULAR) IgG4-RD classification criteria.

Inclusion criteria comprise:

To be included in the study, each individual must satisfy all of the following criteria:
1. Male or female adults, who have reached the age of consent in the applicable region at time of informed consent.
2. Written informed consent and any locally required authorization (e.g., Health Insurance Portability and Accountability Act in the United States [US], European Union [EU] Data Privacy Directive in the EU) obtained from the subject prior to performing any protocol-related procedures, including screening evaluations.
3. Clinical diagnosis of IgG4-RD.
4. Fulfillment of the 2019 ACR/EULAR classification criteria as determined by the Eligibility Committee. Specifically, subjects must meet the classification criteria entry requirements (including involvement of one of the following organs: pancreas, bile ducts/biliary tree, orbits, lungs, kidneys, lacrimal glands, major salivary glands, retroperitoneum, aorta, pachymeninges, or thyroid gland [Riedel's thyroiditis]), must not meet any of the classification criteria exclusions, and must achieve at least 20 classification criteria inclusion points.
5. Experiencing (or recently experienced) an IgG4-RD flare that requires initiation or continuation of GC treatment at the time of informed consent. This criterion may be met in two ways:
   On GC therapy for recent IgG4-RD flare, having received a maximum of 4 weeks of treatment prior to informed consent at a dose no higher than 60 mg/day prednisone or equivalent, and at 20 mg/day prednisone or equivalent on the day prior to randomization, or
   Experiencing active disease not currently being treated at the time of informed consent, with planned initiation of treatment for flare with GC at a maximum dose of 60 mg/day prednisone (or equivalent) and with a plan to be treated at a dose of 20 mg/day of prednisone (or equivalent) on the day prior to randomization, for a total duration of GC treatment during screening of at least 3 weeks at the time of randomization.
   This GC therapy can either be newly initiated or be increased from a maintenance dose of ≤10 mg/day of prednisone or equivalent.
   Subjects unable to be tapered to 20 mg/day of prednisone or equivalent by Visit 2 may not be randomized. Total duration of GC treatment must be at least 3 weeks and not exceed 8 weeks prior to randomization.
6. IgG4-RD affecting at least 2 organs/sites at any time in the course of IgG4-RD with documentation to confirm. One organ must meet the requirements for the ACR/EULAR classification criteria (inclusion 4); the second organ is as defined by the investigator.
7. Willing and able to comply with the protocol, complete study assessments, and complete the study period.
8. Non-sterilized male subjects who are sexually active with a female partner of childbearing potential must use a condom with spermicide from Day 1 through to the end of the study and must agree to continue using such precautions for at least 6 months after the final dose of IP.
   Females of childbearing potential must have a negative serum pregnancy test at screening. Females of childbearing potential who are sexually active with a non-sterilized male partner must use a highly effective method of contraception from signing informed consent and must agree to continue using such precautions through the end of the follow-up of the study and at least 180 days after the last dose of IP; cessation of contraception after this point should be discussed with a responsible physician. Periodic abstinence, the rhythm method, and the withdrawal method are not acceptable methods of contraception. A recommendation will be made that the female partners (of childbearing potential) of male study participants should use a highly effective method of contraception other than a barrier method.
   Females of childbearing potential are defined as those who are not surgically sterile (i.e., surgical sterilization includes bilateral tubal ligation, bilateral oophorectomy, or hysterectomy) or those who are not postmenopausal (defined as 12 months with no menses without an alternative medical cause and a follicle-stimulating hormone within the postmenopausal range as established by the clinical laboratory).

### Exclusion Criteria

Any of the following excludes a subject from participation in the study:
1. Severe cardiovascular, respiratory, endocrine, gastrointestinal, hematological, neurological, psychiatric, or systemic disorder, or any other condition that, in the opinion of the Investigator, would place the patient at unacceptable risk of complications, interfere with evaluation of the IP, or confound the interpretation of patient safety or study results.
2. History of solid organ or cell-based transplantation.
3. Known immunodeficiency disorder.
4. Active malignancy or history of malignancy that was active within the last 10 years, except as follows:
   In situ carcinoma of the cervix following apparently curative therapy >12 months prior to screening,
   Cutaneous basal cell or squamous cell carcinoma following apparently curative therapy,
   Prostate cancer treated with radical prostatectomy or radiation therapy with curative intent >3 years prior to screening and without known recurrence or current treatment, or
   Thyroid cancer for which surgery has been performed and there is no evidence of active disease.
5. Receipt of any biologic B cell-depleting therapy (e.g., rituximab, ocrelizumab, obinutuzumab, ofatumumab, Inebilizumab) in the 6 months prior to screening. Receipt of such a B cell-depleting agent in the period 6-12 months prior to screening.
6. Receipt of non-depleting B-cell-directed therapy (e.g., belimumab), abatacept, or other biologic immunomodulatory agent within 6 months prior screening.
7. Receipt of non-biologic DMARD or immunosuppressive agent other than GCs (e.g., azathioprine, mycophenolate mofetil, methotrexate, others) within 4 weeks prior to screening.
8. Receipt of any investigational agent <12 weeks or <5 half-lives of the drug (whichever is longer) prior to screening.
9. Inability to be tapered off of GC therapy by 8 weeks post-randomization (other than ≤2.5 mg/day prednisone or equivalent for treatment of adrenal insufficiency or intolerance of steroid taper) in the opinion of the Investigator.
10. Receipt of live vaccine or live therapeutic infectious agent within the 2 weeks prior to screening.
11. Pregnancy, lactation, or planning to become pregnant within 6 months of the last dose of IP.
12. Positive test for, or prior treatment for, hepatitis B or HIV infection. A positive test for hepatitis B is detection of either (1) hepatitis B surface antigen (HBsAg); or (2) hepatitis B core antibody (anti-HBc); and in Japan only (3) hepatitis B surface antibody (HBsAb).
13. History of untreated hepatitis C infection, or positive antibody test for hepatitis C virus (HCV) unless patient is considered to be cured following antiviral therapy and has a HCV viral load below the limit of detection at least 24 weeks after completion of treatment at site or central lab.
14. Evidence of active tuberculosis (TB) or being at high risk for TB based on:
   History of active TB or untreated/incompletely treated latent TB. Patients with a history of active or latent TB who have documentation of completion of treatment according to local guidelines may be enrolled.
   History of recent (≤12 weeks of screening) close contact with someone with active TB (close contact is defined as ≥4 hours/week OR living in the same household OR in a house where a person with active TB is a frequent visitor).
   Signs or symptoms that could represent active TB by medical history or physical examination.
   Positive, indeterminate, or invalid interferon-gamma release assay test result at screening, unless previously treated for TB. Patients with an indeterminate test result can repeat the test once, but if the repeat test is also indeterminate, the patient is excluded.
   Chest radiograph, chest computed tomography (CT) or MRI scan that suggests a possible diagnosis of TB or suggests that a work-up for TB should be considered; all patients must have had lung imaging with an acceptable reading within 6 months prior to consent, or during screening.
15. History of >1 episode of herpes zoster (any grade) and/or any other definite or probable opportunistic infection in the 12 months prior to screening for details on opportunistic infections that require exclusion).
16. Known history of allergy or reaction to any component of Inebilizumab formulation or history of anaphylaxis to any human gamma globulin therapy.
17. Allergy to or intolerance of protocol-required treatment, including medications for prophylaxis of infusion reactions (antipyretic such as paracetamol/acetaminophen or equivalent, diphenhydramine or equivalent, and methylprednisolone or equivalent).
18. Estimated glomerular filtration rate <30 mL/min/1.73 m² by Modification of Diet in Renal Disease Study (MDRD) equation.
19. Blood tests at screening that meet any of the following criteria:
   Hemoglobin<7.5 g/dL
   Neutrophils<1200/mm³
   Platelets<110×10⁹/L
   Eosinophil count>3000/mm³
   Prothrombin time>1.2×upper limit of normal (ULN): however subjects who are anticoagulated due to atrial fibrillation and who have aspartate aminotransferase (AST) and alanine aminotransferase (ALT) levels≤2×ULN are not excluded
   Total immunoglobulins<600 mg/dl
   CD19+ B cells at screen <40 cells/uL: an exclusionary value may be repeated
20. Subjects with the following abnormal liver function tests in the absence of hepatobiliary IgG4-RD activity:
   Aspartate aminotransferase (AST)>2×ULN
   Alanine aminotransferase (ALT)>2×ULN
   Total bilirubin (TBL)>2×ULN unless AST, ALT, and hemoglobin are within central laboratory normal range and the patient has a known history of Gilbert syndrome
   Or
   Subjects with the following abnormal liver function tests in the presence of hepatobiliary IgG4-RD activity:

$$AST > 10 \times ULN$$

$$ALT > 10 \times ULN$$

$$TBL > 5 \times ULN$$

Screening liver function tests may be repeated prior to randomization to permit abnormal values due to hepatobiliary IgG4-RD activity to respond to GC treatment.
21. Known positive anti-neutrophil cytoplasmic antibodies (ANCA) targeted against proteinase 3 or myeloperoxidase based on patient records.
22. History of alcohol or drug abuse that, in the opinion of the Investigator, might affect patient safety or compliance with visits or interfere with safety or other study assessments.
23. Active, clinically significant infection at the time of randomization (IP administration may be delayed until recovery, if within screening window, otherwise subject may be rescreened).
24. Participation in any clinical trial that includes use of any pharmacologic intervention.

During the screening period (up to 28 days), all subjects must receive at least 3 weeks of GC treatment for their recent flare. The exact GC dose and taper schedule prior to randomization will be determined by a person of skill, but subjects must be at a dose of 20 mg/day prednisone or equivalent on the day prior to randomization. This GC treatment can either be newly initiated or be increased from a prior maintenance dose of ≤10 mg/day of prednisone or equivalent. The GC treatment for recent flare must not have exceeded 60 mg/day prednisone or equivalent at any point. Patients who are receiving GCs for treatment of recent flare at the time of consent are not eligible if this treatment has exceeded 4 weeks in duration.

### Baseline Characteristics

A complete physical examination (with the exception of rectal and pelvic examinations) will be conducted at baseline, including vital signs, height, and weight.

Blood will be collected for: IgG4 levels, antineutrophil cytoplasmic antibodies (ANCA), Hepatitis B testing: HBsAg, anti-HBc, and in Japan only HBsAb, Hepatitis C antibody (HCV load in patients at least 24 weeks after curative treatment for HCV), HIV testing: HIV-1 antibody, HIV-2 antibody. TB testing (e.g., QuantiFERON®-TB Gold Test), and serum will be frozen for possible testing for JCV antibodies in case of suspected progressive multifocal leukoencephalopathy (PML).

Baseline imaging will be performed unless recent readings are available from within the prior 3 months: CT or MRI scan (or equivalent) of chest, abdomen, and pelvis; and if subject has head or neck disease, an MRI (or equivalent) of the affected area.

### Preparative Regimen

Premedication with a corticosteroid (e.g., methylprednisolone 100 mg IV or equivalent), an antihistamine (e.g., diphenhydramine 25-50 mg orally or equivalent), and an anti-pyretic (e.g., acetaminophen 500-650 mg orally or equivalent) will be administered approximately 30-60 minutes prior to each infusion.

### Glucocorticoid Taper

All subjects will receive daily oral prednisone (or equivalent) from Day 1 through Week 8, according to a tapering dose schedule, Table 2.

TABLE 2
Glucocorticoid Taper Schedule

| Week | Oral Prednisone Dose (or Equivalent) |
|---|---|
| Weeks 1-2 | 20 mg/day |
| Weeks 3-4 | 15 mg/day |
| Weeks 5-6 | 10 mg/day |
| Weeks 7-8 | 5 mg/day |

### Treatment

Inebilizumab group: subjects will receive an infusion of Inebilizumab 300 mg IV on Day 1, Day 15, and at Week 26. Placebo group: Subjects will receive an infusion of placebo on Day 1, Day 15, and at Week 26 according to the dosing schedule of Table 3.

TABLE 3
Summary of Product and Dosing Regimen

| | | Route | Dosing |
|---|---|---|---|
| Product | Concentration and Formulation | Dose | Regimen |
| Inebilizumab | Inebilizumab for IV administration is supplied as a | IV | Day 1, |
| (100 mg per | sterile liquid filled at a nominal volume of 10 mL | 300 mg | Day 15, |
| vial, | in 10R vials. Each vial contains Inebilizumab | | and |
| nominal) | formulated at 10 mg/mL, in 20 mM | | Week 26 |
| | histidine/histidine hydrochloride, 70 mM NaCl, | | |
| | 106 mM (4% [w/v]) trehalose dihydrate, and | | |
| | 0.01% (w/v) polysorbate 80, pH 6.0 | | |
| Placebo | Placebo for IV administration is supplied in 10R | IV | Day 1, |
| | vials filled with 10 mL (nominal) solution | | Day 15, |
| | containing 10 mM histidine/histidine | | and |
| | hydrochloride, 75 mM NaCl, 106 mM (4% [w/v]) | | Week 26 |
| | trehalose dihydrate, and 0.02% (w/v) polysorbate | | |
| | 80, pH 6.0 | | |

Both groups: oral prednisone (or equivalent) tablets from Day 1 to the end of Week 8 (tapering dose regimen: 2 weeks each at 20, 15, 10, and 5 mg/day of prednisone or equivalent, open label, from commercial supply), see FIG. 1. Both treatment groups will receive open-label IV methylprednisolone 100 mg (or equivalent), diphenhydramine 25-50 mg or equivalent, and paracetamol/acetaminophen 500-650 mg or equivalent, 30-60 minutes prior to each IV infusion for prophylaxis of infusion reactions. Both treatment groups will continue to receive open-label GCs, during an 8-week taper regimen (2 weeks each at 20, 15, 10, and 5 mg/day of prednisone or equivalent), representing completion of the GC taper regimen following treatment for their recent flare.

Table 4 exemplifies the infusion rate of Inebilizumab and placebo. Unless an infusion reaction occurs resulting in discontinuation of infusion, the entire infusion bag contents are administered, and the tubing is flushed with a volume of saline at least as large as that of the tubing to ensure complete delivery of the product infused at a rate not to exceed 333 mL/hour. After completion of the infusion, subjects will be observed for at least one hour.

TABLE 4
Exemplary Infusion rate in 250 mL IV bag

| Elapsed Time (minutes) | Infusion Rate (mL/hour) |
|---|---|
| 0-30 | 42 |
| 31-60 | 125 |
| 61 to end of infusion | 333 |

Post-infusion, vital signs of treated subjects are monitored. Exemplary vital signs comprise body temperature, blood pressure, pulse rate, and respiratory rate. Vital signs are collected within 60 min prior to treatment administration, 15 (±5) minutes after the start of the infusion and then every 30 (±10) minutes until completion of infusion, at time of infusion completion, and 60 (±10) minutes after dosing (repeated as needed until stable), as needed.

Optionally, subjects may participate in a one-year follow up period and will receive Inebilizumab. Flare data collection will continue during this period, and the flare rate will be calculated. The optional period will also contribute to the safety database of Inebilizumab in patients with IgG4-RD by increasing the number of patients exposed (those who originally received placebo and who then receive Inebilizumab) as well as the duration of exposure.

### Post-Treatment Assessment

### IgG4-RD Responder Index

The IgG4-RD RI is a validated instrument for longitudinal assessment of IgG4-RD disease activity, developed and refined by an international team of IgG4-RD experts specifically to permit structured assessment of response to treatment by clinical investigators (Carruthers et al, 2012, Wallace et al, 2018). The RI captures disease activity and damage in 25 domains, with higher weights for disease manifestations that require urgent treatment or that worsen despite treatment. The RI incorporates an activity score for each organ/site of disease, a score for symptomatic disease at each of these sites, and a score amendment for a need for urgent treatment. Serum IgG4 concentration and presence of organ-specific damage are collected but are not part of the overall RI.

TABLE 5
Randomized-Controlled Period Assessments and Procedures

| | Study Period | | | | | | | | | | | | | |
|---|---|---|---|---|---|---|---|---|---|---|---|---|---|---|
| | Randomized-Controlled Period | | | | | | | | | | | | | |
| | Visit Number | | | | | | | | | | | | | |
| | V2 | V3 | V4 | V5 | V6 | V7 | V8 | V9 | V10 | V11 | V12 | V13 | V14 | V15 |
| | Week | | | | | | | | | | | | | |
| | | | | | | | | | | | | | | 52 or |
| | 0 | 2 | 4 | 8 | 12 | 16 | 20 | 26 | 30 | 34 | 38 | 42 | 46 | EDV ^{d} |
| | Nominal Day | | | | | | | | | | | | | |
| | 1 | 15 | 29 | 57 | 85 | 113 | 141 | 183 | 211 | 239 | 267 | 295 | 323 | 365 |
| | Window | | | | | | | | | | | | | |
| | | ±1 | ±3 | ±3 | ±3 | ±5 | ±5 | ±5 | ±7 | ±7 | ±7 | ±7 | ±7 | ±7 |
| | — | day | days | days | days | days | days | days | days | days | days | days | days | days |
| Assessment of | X | X | X | X | X | X | X | X | X | X | X | X | X | X |
| AEs/SAEs/AESIs | | | | | | | | | | | | | | |
| Concomitant | X | X | X | X | X | X | X | X | X | X | X | X | X | X |
| medications | | | | | | | | | | | | | | |
| Physician Global | X | X | X | X | X | X | X | X | X | X | X | X | X | X |
| Assessment of | | | | | | | | | | | | | | |
| Disease Activity | | | | | | | | | | | | | | |
| Completion of PROs | X | X | X | X | X | X | X | X | X | X | X | X | X | X |
| (SF-36v2, | | | | | | | | | | | | | | |
| FACIT-Fatigue) | | | | | | | | | | | | | | |
| and PGA ^{a} | | | | | | | | | | | | | | |
| ECG (prior to IP | X | | | | | | | X | | | | | | X |
| administration) | | | | | | | | | | | | | | |
| Vital Signs | X ^{b} | X ^{b} | X | X | X | X | X | X ^{b} | X | X | X | X | X | X |
| Weight | X | | | | | | | X | | | | | | X |
| Full physical | X | | | | | | | X | | | | | | X |
| examination | | | | | | | | | | | | | | |
| Symptom-driven physical | | X | X | X | X | X | X | | X | X | X | X | X | |
| exam | | | | | | | | | | | | | | |
| IgG4-RD RI | X | X | X | X | X | X | X | X | X | X | X | X | X | X |
| Urine pregnancy test in | X | X | X | X | X | X | X | X | X | X | X | X | X | X |
| women capable of | | | | | | | | | | | | | | |
| pregnancy | | | | | | | | | | | | | | |
| Urinalysis (microscopic | X | X | X | X | X | X | X | X | X | X | X | X | X | X |
| if abnormal) and urine | | | | | | | | | | | | | | |
| protein/creatinine | | | | | | | | | | | | | | |
| ratio | | | | | | | | | | | | | | |
| Safety labs (chemistry, | X | X | X | X | X | X | X | X | X | X | X | X | X | X |
| hematology, coagulation) | | | | | | | | | | | | | | |
| Inebilizumab PK ^{c} | X | X | X | X | X | | | X | X | | X | | | X |
| ADA | X | | X | | X | | | X | | | X | | | X |
| Complement levels | X | X | X | X | X | X | X | X | X | X | X | X | X | X |
| (C3, C4) | | | | | | | | | | | | | | |
| ELF score | X | | | | | | | X | | | | | | X |
| IgG4 and IgE levels | X | X | X | X | X | X | X | X | X | X | X | X | X | X |
| SPEP | X | | | | | | | | | | | | | X |
| Total Ig, IgA, IgM, | X | | | | X | | | X | | | X | | | X |
| IgG, and IgG | | | | | | | | | | | | | | |
| subclasses | | | | | | | | | | | | | | |
| Whole blood for B | X | X | X | X | X | | X | X | | X | X | | X | X |
| cell flow cytometry | | | | | | | | | | | | | | |
| Exploratory biomarkers | X | X | X | X | X | | X | X | | X | X | | X | X |
| (plasma and serum) | | | | | | | | | | | | | | |
| RNA PAXgene tube (for | X | X | X | X | X | | X | X | | X | X | | X | X |
| gene expression; not | | | | | | | | | | | | | | |
| obtained in China) | | | | | | | | | | | | | | |
| DNA PAXgene (optional, | X | | | | | | | | | | | | | |
| with consent) | | | | | | | | | | | | | | |
| Verify eligibility | X | | | | | | | | | | | | | |
| Randomization | X | | | | | | | | | | | | | |
| IP administration ^{e} | X | X | | | | | | X | | | | | | |
| Provide oral GCs | X | | | | | | | | | | | | | |
| Assess GC compliance | | X | X | X | | | | | | | | | | |

AE = adverse event; ADA = anti-drug antibodies; AESI = adverse event of special interest; ECG = electrocardiogram; EDV = early discontinuation visit; ELF = enhanced liver fibrosis score; FACIT-Fatigue = Functional Assessment of Chronic Illness Therapy-Fatigue; GC = glucocorticoid; Ig = immunoglobulin(s); IgG4-RD RI = IgG4-RD Responder Index; IP = investigational product; PGA = patient global assessment; PK = pharmacokinetic; PRO = patient-reported outcome; SAE = serious adverse event; SF-36v2 = 36-Item Short Form Survey Version 2 (Acute Recall) Questionnaire; SPEP = serum protein electrophoresis; V = visit.
Note:
On days of IP administration, all procedures and blood sampling, except for post-dose PK, must be collected before IP administration.
^{a} Subjects should complete all PRO assessments prior to other procedures.
^{b} Vital signs
^{c} On infusion IP dosing days, Inebilizumab PK serum samples are to be collected predose and approximately 15 minutes (±5 minutes) after completion of the infusion. On nondosing days, only 1 PK serum sample is required.
^{d} Subjects who have not had at least 90 days of follow-up after their last dose of Inebilizumab but are have indicated their intent to withdraw consent for participation in the RCP should be asked if they would be willing to have an EDV for final safety follow-up at 90 days after final Inebilizumab dose.
^{e} Premedication will be administered approximately 30-60 minutes prior to each IP infusion.

TABLE 6
Open-Label Period Assessments and Procedures

| | Study Period | | | | | | | | | | | | |
|---|---|---|---|---|---|---|---|---|---|---|---|---|---|
| | Open-Label Period | | | | | | | | | | | | |
| | OLP Visit Namber | | | | | | | | | | | | |
| | | | | | | | | | | | OLP V | OLP | |
| | OLP | OLP | OLP | OLP | OLP | OLP | OLP | OLP | OLP | OLP | 15, 17, | V16, | OLP |
| | V1 | V2 | V3, V4 | V5 | V6, V7 | V8 | V9, V10 | V11 | V12, V13 | V14 | 19, 21 | 18, 20 | V22/EDV |
| | OLP Week(s) | | | | | | | | | | | | |
| | | | | | | | | | | | 61, 87, | 78, 104, | |
| | 0 | 2 | 4, 8 | 12 | 16, 20 | 26 | 30, 34 | 38 | 42, 46 | 52 | 113, 139 | 130 | 156 |
| | Window | | | | | | | | | | | | |
| | | | Phone | | Phone | | Phone | | Phone | | Phone | | |
| | 1-38 days | | contact | | contact | | contact | | contact | | contact | | |
| | after last | ±2 | ±10 | ±10 | ±10 | ±10 | ±10 | ±10 | ±10 | ±10 | ±20 | ±20 | ±20 |
| | RCP visit | days | days ^{b} | days | days | days | days ^{b} | days | days ^{b} | days | days ^{b} | days | days |
| Assessment of | X ^{c} | X | X | X | X | X | X | X | X | X | X | X | X |
| AEs/SAEs/AESIs | | | | | | | | | | | | | |
| Concomitant | X | X | X | X | X | X | X | X | X | X | X | X | X |
| medications | | | | | | | | | | | | | |
| Physician Global | X | X | | X | | X | | X | | X | | X | X |
| Assessment of | | | | | | | | | | | | | |
| Disease Activity | | | | | | | | | | | | | |
| Vital signs | X ^{d} | X ^{d} | | X | | X ^{d} | | X | | X | | X | X |
| Physical exam | X | | | | | | | | | X | | | X |
| Symptom-driven | X | X | | X | | X | | X | | X | | X | |
| physical exam | | | | | | | | | | | | | |
| IgG4-RD RI | X | X | | X | | X | | X | | X | | X | X |
| Urine pregnancy test | X | X | | X | | X | | X | | X | | X | X |
| in women capable of | | | | | | | | | | | | | |
| pregnancy ^{e} | | | | | | | | | | | | | |
| Safety labs ^{f} | X | X | | X | | X | | X | | X | | X | X |
| ELF score | X | | | | | X | | | | X | | X | X |
| Urinalysis ^{g} | X | X | | X | | X | | X | | X | | X | X |
| ADA | X | X | | X | | X | | X | | X | | X | X |
| IgG4 and IgE | X | X | | X | | X | | X | | X | | X | X |
| Total immunoglobulins, | X | | | | | X | | | | X | | X | X |
| IgA, IgM, IgG, and | | | | | | | | | | | | | |
| IgG subclasses | | | | | | | | | | | | | |
| B cell flow cytometry | X | | | X | | X | | X | | X | | X | X |
| panel | | | | | | | | | | | | | |
| RNA PAXgene tube | X | | | X | | X | | X | | X | | X | X |
| (for gene expression; | | | | | | | | | | | | | |
| Not obtained in China) | | | | | | | | | | | | | |
| Assessment of | X | | | | | | | | | | | | |
| eligibility for OLP | | | | | | | | | | | | | |
| IP administration ⁱ | X | X | | | | X | | | | | | X | |

AE = adverse event; ADA = anti-drug antibodies; AESI = adverse event of special interest; EDV = early discontinuation visit; ELF = enhanced liver fibrosis score; Ig = immunoglobulin; IgG4-RD RI = IgG4-RD Responder Index; IP = investigational product, OLP = open-label period; RCP = randomized-controlled period; SAE = serious adverse event; SPEP = serum protein electrophoresis; V = visit.
Note:
Procedures and blood sampling is collected before IP administration on days when IP is administered.
^{a} Subjects who have received immunosuppressive therapy other than glucocorticoid treatments must have a washout period of at least 28 days prior to Dose 1 of Inebilizumab in the OLP.
^{b} Monthly telephone calls in months without in-person visits.
^{c} AEs and SAEs occurring after completion of the RCP must be collected at OLP Visit 1.
^{d} Vital signs obtained.
^{e} Perform on days of dosing; confirm negative prior to dose.
^{f} Safety labs include chemistry, hematology, and coagulation; SPEP will not be performed during the OLP.
^{g} Microscopic if abnormal.
^{h} Premedication will be administered approximately 30-60 minutes prior to each IP infusion.

### Assessment of Flare

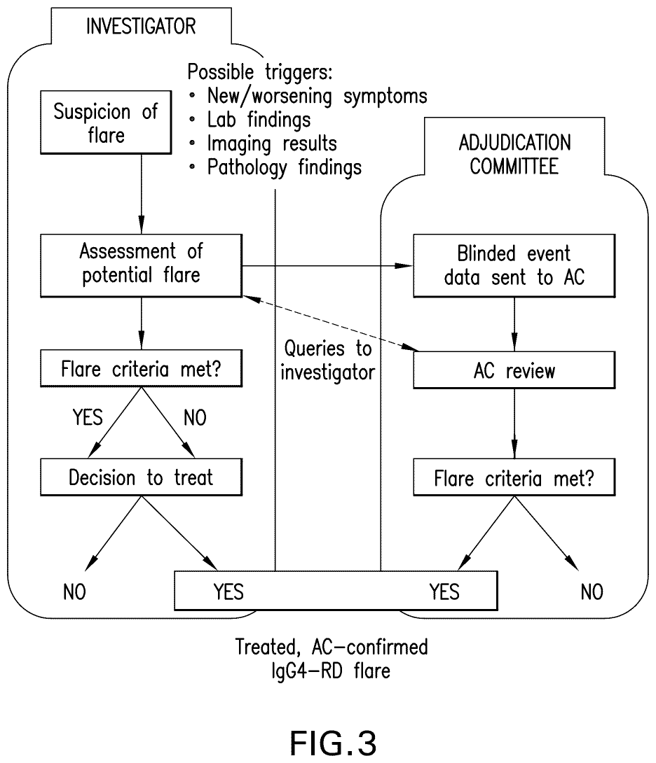

Data to be evaluated when considering a potential flare may include: symptoms, physical examination findings, laboratory results, imaging results, and biopsy results. An exemplary schematic showing flare assessment workflow is shown in FIG. 3.

### IgG4-RD Flare

In embodiments, an IgG4-RD flare comprises new or worsening signs and symptoms of IgG4-RD disease activity that meet one or more organ-specific flare criteria. In embodiments, there may be no clear alternative diagnosis or conflicting biopsy findings. Exemplary flare criteria are described below:

For each potential flare event, the investigator provides: (1) observation that led to initiation of evaluation for flare: patient-reported new/worsening symptoms, new/worsening physical exam finding, new/worsening laboratory finding, incidental finding on imaging: (2) organ-specific findings: findings are recorded separately for every affected organ, even if flare criteria for that organ are not met. Data provided include symptoms, physical exam findings, laboratory findings, imaging findings, and biopsy results.

Treatment for flare comprises: glucocorticoids, supportive therapy (e.g., pancreatic enzyme replacement, bile acid sequestrate, artificial saliva/lubricant) immunosuppression other than glucocorticoids, and surgical intervention or other procedural intervention (e.g., stenting).

Pachymeninges findings consisting with pachymeningeal flare: present (1) and either 2a or 2b: (1) new or worsening symptom or PE finding consistent with IgG3-RD pachymeningitis; (2a) CSF lab finding consistent with IgG4-RD pachymeningitis; or (2b) imaging finding confirming new or worsening meningeal involvement. Absent=alternative diagnosis or inconsistent biopsy finding.
a. Symptoms: Headache.
b. Physical exam: CN palsy, neurologic abnormalities consistent with radiculomyelopathy.
c. Laboratory: CSF pleocytosis and CSF increased protein.
d. Imaging: Meningeal enhancement or thickening.
e. Biopsy: consistent with pachymeningeal flare.

Pituitary gland findings consisting with pituitary flare: at least one of the following are present: either new or worsening anterior/posterior endocrine dysfunction in subject with known pituitary disease or imaging finding confirming new or worsening pituitary involvement. An alternative diagnosis or inconsistent biopsy finding is absent.
a. Symptoms: Visual field abnormalities, headache, symptoms consistent with anterior or posterior pituitary endocrine failure.
b. Physical exam: CN palsy, visual field abnormalities.
c. Laboratory: Pituitary endocrine dysfunction.
d. Imaging: Pituitary mass or enhancement.
e. Biopsy: consistent with pituitary gland flare.

Orbit finding consistent with flare in orbits: at least one of the following is present: either new or worsening symptom or PE finding consistent with orbital flare or new or worsening orbital abnormality on imaging consistent with orbital flare. An alternative diagnosis or inconsistent biopsy finding is absent.
a. Symptoms: Diplopia, proptosis, foreign body sensation, eye or retrobulbar discomfort or pain, or other visual symptoms including vision blurring or loss, symptoms consistent with scleritis, symptoms from compression of peripheral nerves in the area of the orbit, such as trigeminal and infra-orbital nerves (pain or numbness).
b. Physical exam: Proptosis, supra-orbital swelling or other peri-orbital swelling consistent with enlargement of extra-ocular muscles, field cuts, cranial nerve palsies, extraocular movement abnormality, infra-orbital/supra-orbital nerve enlargement.
c. Imaging: Compatible with orbital disease (enlargement of extra-ocular muscles, enlargement of optic nerve including abnormalities of retrobulbar space or within cavernous sinus).
d. Biopsy: consistent with orbital flare.

Lacrimal Glands findings consistent with flare in lacrimal glands: new or worsening lacrimal gland enlargement/mass on PE or observed on imaging. An alternative diagnosis or inconsistent biopsy finding is absent.
a. Symptoms: Lacrimal gland discomfort, pain or swelling; redness of the eye, excessive tearing; eyelid crusting; blurred vision.
b. Physical exam: Lacrimal gland swelling/mass.
c. Imaging: Consistent with lacrimal gland swelling.
d. Biopsy: consistent with lacrimal gland flare.

Salivary Glands findings consistent with salivary gland flare: Present: new or worsening salivary gland enlargement or tenderness on PE or new or worsening enlargement on imaging. An alternative diagnosis or inconsistent biopsy finding is absent.
a. Symptoms: Pain or swelling of gland(s).
b. Physical exam: Salivary gland swelling or tenderness.
c. Imaging: Compatible with salivary gland swelling.
d. Biopsy: consistent with salivary gland flare.

Lymph node(s) findings consistent with flare in lymph node(s): At least one of the following is present: (a) in a subject with concurrent IgG4 disease in another organ: multiple enlarged nodes (primarily nontender) by PE or imaging in an area separate from other current organ with flare; or (b) in a subject with no other organ with concurrent flare: multiple enlarged lymph nodes (primarily nontender) by PE or imaging and lymph node biopsy to exclude other diagnosis, such as a malignancy. An alternative diagnosis or inconsistent biopsy finding is absent.
a. Symptoms: Subject reported swelling.
b. Physical exam: Lymphadenopathy
c. Imaging: Consistent with lymphadenopathy.
d. Biopsy: consistent with lymph node flare.

Lungs, Including Pleura and Parenchyma findings consistent with flare in lymph node(s): typically new or worsening imaging finding confirming pleuropulmonary involvement. An alternative diagnosis or inconsistent biopsy finding is absent.
a. Symptoms: Dyspnea at rest or with exertion, cough.
b. Physical exam: Increased respiratory rate, findings suggestive of pleural effusion, dry crackles compatible with pulmonary fibrosis, localized diminished breath sounds, findings consistent with infiltrate.
c. Laboratory: New or worsening pulmonary function test abnormalities consistent with lung flare.
d. Imaging: Pulmonary nodules or mass and/or pulmonary infiltrate/ground glass opacities consistent with interstitial pneumonia and/or pulmonary fibrosis and/or pleural effusion or pleural thickening and/or peribronchovascular and septal thickening and/or paravertebral mass, paravertebral band-like soft tissue in thorax.
e. Biopsy: consistent with lung flare.

Aorta & large blood vessels findings consistent with aorta & large blood vessels flare: at least one of the following is present: (A) new or worsening aortic or other vessel wall thickening or other evidence of aortitis (dissection, aneurysm) by imaging; or (b) demonstration of new or worsening aortitis, dissection or aneurysm or similar findings for other large artery at surgery or intervention (stenting) or an aneurysm, dissection, or other vascular anomaly.
a. Symptoms: Pain, palpable mass and systemic/constitutional.
b. Physical exam: Palpable arterial mass, especially if pulsatile, or bruit.
c. Imaging: Consistent with aneurysm, dissection, thickening/enhancement of vessel wall or other vessel abnormality.
d. Biopsy: consistent with aorta/large blood vessel flare.

Retroperitoneum, Mediastinum, & Mesentery findings consistent with retroperitoneum, mediastinum & mesentery flare: at least one of the following is present: (a) new or worsening imaging evidence of involvement of retroperitoneum, mediastinum, and/or mesentery; or (b) tissue confirmation at time of surgery or intervention (stenting) that confirms new or worsening involvement of retroperitoneum, mediastinum, and/or mesentery. An alternative diagnosis or inconsistent biopsy finding is absent.
a. Symptoms: Pain (e.g., flank, back, thighs, abdominal, other including chronic pain), leg edema, dyspnea, cough.
b. Physical exam: Palpable mass or findings consistent with superior vena cava syndrome, leg edema, or fibrosing mediastinitis.
c. Laboratory: For retroperitoneal involvement of ureters: elevated creatinine, decreased estimated glomerular filtration rate (eGFR).
d. Imaging: Finding of mass lesion, ureteral stenosis or hydronephrosis, findings consistent with superior vena cava syndrome, other evidence of inflammation in retroperitoneum typically with enhancement (often infra-renal, peri-aortic distribution extending down to iliac vessels but may involve root of mesentery), circumferential/antero-lateral soft tissue around infrarenal aorta or iliac arteries, other radiologic evidence of inflammation in mesentery or mediastinum.
e. Biopsy: consistent with retroperitoneum, mediastinum & mesentery flare.

Pancreas and Common Bile Duct findings consistent with flare of pancreas/common bile duct: typically in a subject with a prior history of IgG4-related autoimmune pancreatitis either: (A) new or worsening symptom and/or PE finding and new or worsening laboratory finding consistent with flare in disease of pancreas/common bile duct: or imaging or endoscopic finding that confirms new or worsening involvement of pancreas/common bile duct. In a subject with no prior history of IgG4-related autoimmune pancreatitis: new symptom, PE finding and/or laboratory finding consistent with involvement of pancreas/common bile duct, and either: (a) new imaging or endoscopic finding that confirms involvement of the pancreas/common bile duct, or (b) biopsy evidence of involvement of the pancreas. An alternative diagnosis or inconsistent biopsy finding is absent.
a. Symptoms: Pain (e.g., flank, back, abdominal), weight loss and systemic/constitutional.
b. Physical exam: Abdominal tenderness, jaundice, palpable mass, weight loss.
c. Laboratory: Elevated bilirubin, alkaline phosphatase, GGT, amylase and/or lipase; other, including low fecal elastase, high glucose/HbA1C (describe).
d. Imaging: Consistent with pancreatic mass or diffuse pancreatic enlargement with loss of lobulations, diffuse pancreatic enlargement, pseudocapsule, pancreatic duct stricture, common bile duct abnormality.
e. Biopsy: consistent with pancreatic/common bile duct flare.

Biliary tree (IgG4-RD sclerosing cholangitis) findings consistent with flare of bile ducts/biliary tree: In a patient with a prior history of IgG4-related sclerosing cholangitis, either (a) New or worsening laboratory finding consistent with biliary tree flare; or (b) New or worsening imaging or endoscopic finding that confirms worsening involvement of bile ducts/biliary tree. In a subject with no prior history of IgG4-related sclerosing cholangitis: (a) new laboratory finding consistent with biliary tree flare; and (b) Imaging or endoscopic finding that confirms involvement of the bile ducts/biliary tree. An alternative diagnosis or inconsistent biopsy finding is absent.
a. Symptoms: Itching, abdominal pain, right upper quadrant pain and systemic/constitutional.
b. Physical exam: Abdominal tenderness, jaundice, right upper quadrant fullness.
c. Laboratory: Elevated bilirubin, ALT/AST, alkaline phosphatase, GGT.
d. Imaging: Thickening, mass, strictures, dilatation of extra-hepatic and/or intrahepatic bile ducts.
e. Biopsy of liver or biliary tree: consistent with bile duct/biliary tree flare.

Kidney findings consistent with kidney flare: In a subject with a prior history of IgG4-related renal disease either: (a) worsening renal function or proteinuria; or (b) any imaging finding or biopsy that confirms worsened involvement of the kidney. In a subject with no prior history of IgG4-related renal disease either: (a) worsening renal function, or proteinuria; and either biopsy or imaging finding consistent with renal flare, or (b) worsening renal function in the setting of active IgG4-RD in other organs with worsened hypocomplementemia and increased IgG4. An alternative diagnosis or inconsistent biopsy finding is absent.
a. Symptoms: Fatigue, mental status changes and systemic/constitutional.
b. Physical exam: Edema.
c. Laboratory: Elevated creatinine, decreased eGFR, hematuria or proteinuria; other, including hypocomplementemia.
d. Imaging: Diffuse kidney enlargement, renal abnormality including, hypodense lesions in the renal cortex, renal atrophy, and/or pelvis thickening.
e. Biopsy: consistent with kidney disease flare.

Skin findings consistent with flare of skin disease: present are new or worsening IgG4-RD skin lesions from symptoms or PE and either (a) prior biopsy proven IgG4-related skin disease, or (b) current biopsy consistent with diagnosis. An alternative diagnosis or inconsistent biopsy finding is absent.
a. Symptoms: Rash.
b. Physical exam: Erythematous papules or nodules, hyperpigmented lesions or other skin lesions.
c. Biopsy: consistent with skin disease flare.

Other sclerosis/mass formation in thyroid (Riedel's thyroiditis), liver, breast, prostate, maxillary sinus, nasal septum, pericardium, peripheral nerves, other. At least one of the following is present: (a) new or worsening imaging result that confirms involvement; or (b) any new biopsy evidence of new or worsening involvement. An alternative diagnosis or inconsistent biopsy finding is absent.
a. Symptoms consisting with flare of other sclerotic mass forming disease.
b. Physical exam findings consistent with flare of other sclerotic mass forming disease.
c. Laboratory findings consistent with flare of other sclerotic mass forming disease.
d. Imaging findings consistent with flare of other sclerotic mass forming disease.
e. Biopsy results consistent with flare of other sclerotic mass forming disease.

### Laboratory Assessments

Hematology: A hematology panel will include a complete blood count, with white blood cell count (WBC) and differential (basophils, eosinophils, lymphocytes, monocytes, and neutrophils), hemoglobin, hematocrit, and platelet count.

Chemistry: Serum chemistry will include:

| | |
|---|---|
| Albumin | Immunoglobulins: Total, IgA, IgG, IgE, |
| Alkaline phosphatase (ALP) | IgM, and IgG subclasses |
| Alanine transaminase (ALT) | Lipase |
| Amylase | Magnesium |
| Aspartate transaminase (AST) | Phosphorus |
| Bicarbonate | Potassium |
| Blood urea nitrogen (BUN) | Serum protein electrophoresis (SPEP) |
| Calcium | Serum to be frozen for possible future |
| Chloride | safety testing |
| Creatinine | Sodium |
| Gamma-glutamyl transferase | Total bilirubin (TBL): (if > 1.5 ULN, |
| (GGT) | indirect and direct bilirubin will be |
| Glucose (random) | measured) |
| Hemoglobin A1C (HbA1C) | Total protein |
| | Uric acid |

Additional tests for AST, ALT, ALP, and TBL may be performed.

Coagulation: prothrombin time, international normalized ratio, and activated partial thromboplastin time.

Urinalysis: Urinalysis will evaluate color, appearance, and specific gravity. Standard dipstick analysis, including pH, protein, glucose, blood, and bilirubin, will be performed. Samples with abnormal dipstick will have microscopy performed. Microscopy will include WBC/high power field (HPF) and red blood cell count/HPF. Urine protein/creatinine ratio calculates the protein/creatinine ratio from a random urine sample to estimate the 24-hour protein excretion as a marker of renal disease.

Progressive Multifocal Leukoencephalopathy: In a case of suspected PML, a cerebrospinal fluid sample will be collected to test for JCV by polymerase chain reaction testing using a validated assay in a clinical testing laboratory.

### Biomarker Assessment

Immunoglobulin Levels: Blood will be collected to assess levels of immunoglobulins (IgG, IgG subclasses including IgG4, IgM, IgA, IgE, and total immunoglobulins).

Complement Components: Serum levels of C3 and C4 will be measured. These may also be markers of disease activity.

Flow Cytometry: Peripheral B cells (overall and subsets) will be analyzed by validated flow cytometry assays to assess Inebilizumab PD effects and as potential markers of disease activity.

Serum and Plasma: Serum and plasma will be collected to measure changes in exploratory biomarkers of disease activity, changes in biomarkers related to the Inebilizumab mechanism of action, and changes in biomarkers as a result of Inebilizumab administration. These include, but are not limited to: Serum biomarker expression (e.g., inflammation-related cytokines/chemokines), Enhanced Liver Fibrosis (ELF) score, which represents a composite result from a group of serum markers that identify a quantifiable level of liver fibrosis; however, it may be also used as an outcome measure in patients with IgG4-RD-associated fibrosis, and assessment of autoimmune antibodies to understand their contribution to disease pathogenesis.

RNA and DNA: Blood RNA will be used to measure the plasma cell gene signature (a validated measure of plasma cell abundance in peripheral blood [Streicher et al, 2014]), and expression levels of genes associated with disease activity. DNA may be isolated at baseline from blood to test for polymorphisms in genes relevant to the mechanism of action of Inebilizumab.

Imaging: Chest, abdomen, and pelvis imaging (CT scans, unless a CT scan is contraindicated) at baseline unless a CT scan or other appropriate imaging (such as, but not limited to, positron emission tomography [PET] scan or MRI) had been performed within the previous 3 months and the reading is available. If clinically indicated, imaging (PET scan excluded) may be repeated prior to randomization, to clarify illness, to assess response to GCs. or to ensure exclusion of malignancy.

Immunogenicity: Blood samples for immunogenicity (ADA to Inebilizumab) will be obtained prior to IP administration and will be assessed using a validated immunoassay.

Pharmacokinetic: Blood samples to determine the concentration of Inebilizumab will be taken and measured using a validated immunoassay.

Pharmacodynamic: The effect of Inebilizumab on peripheral B cell counts will be assessed over time using a qualified immunoassay.

### Cumulative Glucocorticoid Use

Cumulative GC use will be calculated as the total GC dose in mg of prednisone or prednisone equivalent taken for the purpose of IgG4-RD disease control.

### Physician and Patient Global Assessments of Disease Activity

The Physician Global Assessment of Disease Activity and Patient Global Assessment of Disease Activity (PGA) are scales used in the assessment of rheumatologic diseases (Rohekar and Pope, 2009). For the physician's global assessment. Investigators will indicate the patient's level of disease activity due to IgG4-RD today, from no disease activity to maximum disease activity, on a 100 mm visual analog scale (VAS). For the PGA, the subject will be asked to rate their current quality of life by making a mark on a 100 mm VAS, labeled “very poorly” to “very well”, in response to these instructions: Considering all the ways that your IgG4-related disease affects you, please rate how well you are doing today.

### Subject-Reported Outcome (Patient-Reported Outcome)

Subject-reported outcomes are measured using 2 instruments: the 36-Item Short Form Survey Version 2 (Acute Recall) Questionnaire (SF-36v2) and the Functional Assessment of Chronic Illness Therapy-Fatigue (FACIT-Fatigue) Scale. The FACIT-Fatigue Scale is a 13-item subject-completed questionnaire used to assess the impact of fatigue (FACIT). Its recall period is 7 days. The responses range from 0 (Not at all) to 4 (Very much). The FACIT-Fatigue Scale is widely used in rheumatoid arthritis clinical studies and is reliable, validated, and sensitive to change (Orbai and Bingham, 2015: Cella et al, 2005). Fatigue can be a prominent complaint in patients with IgG4-RD (Kamisawa et al, 2015).

### Safety Assessment

Clinically important abnormalities in vital signs, body weight, physical examination, laboratory parameters, and ECGs are recorded. Vital signs, including systolic and diastolic blood pressure (mmHg), pulse rate (beats/min), respiratory rate (breaths/min), body temperature (° C.), and body weight (kg), are measured using clinically acceptable methods and devices. Subjects should be seated or supine when vital signs are obtained. Depending on the visit, physical examinations will be either a full examination (excluding rectal and pelvic examinations) or a symptom-driven examination. Examination of the head and neck, including orbits, salivary glands, and lymph nodes, are to be included in both the complete and the symptom-driven physical examinations.

Electrocardiogram: A 12-lead ECG is performed. All ECG recordings will be made with the subject in a supine position, having rested in this position for at least 5 minutes before the start of the ECG. Each ECG includes ventricular heart rate and intervals (PR, RR, QRS, QT, QTc).

Pregnancy: Serum β-hCG pregnancy test(s) is completed for females during the screening period and urine pregnancy tests will be completed in females of childbearing potential at all subsequent study visits. At visits where an IP infusion will be administered, a negative urine pregnancy test result must be obtained prior to administration of IV IP in female patients capable of pregnancy.

### Analysis

### Analysis of the Primary Efficacy Endpoint

The primary efficacy analysis will use the treatment policy strategy, which includes all data captured during the 52-week randomized control period. The primary efficacy variable is the time in days from Day 1 (dosing) to the date of the first treated and determined IgG4-RD flare within the 52-week randomized control period. The date of disease flare is defined as the date of initiation of any flare treatment (new or increased GC treatment, other immunotherapy, or interventional procedure) deemed necessary for the flare. The hazard rate in the Inebilizumab group will be compared to that in the placebo group using the Cox proportional hazards model with the treatment indicator (Inebilizumab or placebo) and the stratification factor as the explanatory variables. The hazard ratio (HR) of Inebilizumab versus placebo will be estimated together with its associated 95% confidence interval (CI). SAS PROC PHREG will be used for fitting this model. The data cutoff for the primary analysis will be when all subjects have completed or discontinued from the randomized control period.

Subjects who discontinue prematurely from the randomized control period without a treated and flare will be censored in this model at the time of discontinuation. Additional analysis with the same model will be performed with data from subjects who meet the following criteria, censored at the time the relevant treatment was first received: (a) received any treatment for IgG4-RD, including GC or immunosuppressive treatment, prior to experiencing a treated and AC-determined flare; or (b) received any prohibited GC or immunosuppressive treatment prior to experiencing a treated and determined flare.

### Analysis of Secondary Efficacy Endpoints

### Annualized Flare Rate for Treated and Determined Flares

The annualized flare rate for treated and determined flares during the randomized control period is analyzed with a treatment policy strategy, which includes all data captured during the 52-week randomized control period. The efficacy variable is annualized flare rate, which is compared between the Inebilizumab group and the placebo group using a negative binomial model. The response variable in the model is the number of treated determined flares experienced by a subject over the 52-week randomized control period. The model will include covariates of treatment group (Inebilizumab or placebo) and stratification factor. The logarithm of the subject's corresponding follow-up time is used as an offset variable in the model to adjust for patients having different exposure times during which the events occur. The estimated treatment effect (i.e., the rate ratio of Inebilizumab versus placebo), corresponding 95% CI, and two-sided p-value for the rate ratio is presented. In addition, the annual flare rate and the corresponding 95% CI within each treatment group, and the absolute difference between treatment groups with the corresponding 95% CI, is presented.

### Annualized Flare Rate for Flares During the Randomized Control Period, Whether or not Treated

The annualized flare rate determined flares, whether or not treated, during the randomized control period is analyzed with a treatment policy strategy, which includes all data captured during the 52-week randomized control period. The efficacy variable is the annualized flare rate, which is compared between the Inebilizumab group and the placebo group using a negative binomial model. The response variable in the model is the number of AC-determined flares, regardless of treatment experienced by a subject, over the 52-week randomized control period. The model will include covariates of treatment group (Inebilizumab or placebo) and stratification factor. The logarithm of the subject's corresponding follow-up time is used as an offset variable in the model to adjust for patients having different exposure times during which the events occur. The estimated treatment effect (i.e., the rate ratio of Inebilizumab versus placebo), corresponding 95% CI, and two-sided p-value for the rate ratio is presented. In addition, the annual flare rate and the corresponding 95% CI within each treatment group, and the absolute difference between treatment groups with the corresponding 95% CI, is presented.

### Flare-Free Complete Remission

The analysis of the proportion of subjects achieving flare-free complete remission at Week 52 for the composite estimand will include all data captured during the 52-week randomized control period. Complete remission is defined as an IgG4-RD Responder Index score of 0 at Week 52, no AC-determined flare during the randomized control period, and no treatment for flare or disease control during the randomized control period except the required 8-week GC taper. The efficacy variable is the proportion of subjects achieving flare-free complete remission at Week 52, which is assessed using a logistic regression model. The response variable in the model is whether or not a subject achieves flare-free complete remission at Week 52. The model will have treatment indicator (Inebilizumab or placebo) and stratification factor as the explanatory variables. Subjects who discontinue prematurely from the randomized control period is treated as not achieving complete remission at Week 52. The results of the analyses is presented using odds ratios, together with associated 95% CI and two-sided p-value for each active dose regimen versus placebo. An additional analysis under a treatment policy strategy with the same model is conducted, with complete remission defined as an IgG4-RD RI score of 0 at Week 52 and no determined flare during the randomized control period.

### Time to Initiation of Treatment for IgG4-RD. Regardless of Determination of Flare

The analysis of time to initiation of treatment for IgG4-RD activity by the Investigator within the randomized control period, regardless of determination of flare, will use a treatment policy strategy, which includes all data captured during the 52-week randomized control period The date of disease flare is defined as the date of initiation of any flare treatment (new or increased GC treatment, other immunotherapy, or interventional procedure) deemed necessary by the Investigator for the flare. The efficacy variable is the time in days from Day 1 (dosing) to the date of the first treatment for disease activity by the Investigator within the randomized control period, regardless of AC determination of flare within the 52-week randomized control period. The hazard rate in the Inebilizumab group is compared to that in the placebo group using the Cox proportional hazards model with the treatment indicator (Inebilizumab or placebo) and the stratification factor as the explanatory variables. The HR of Inebilizumab versus placebo is estimated together with its associated 95% CI. SAS PROC PHREG is used for fitting this model. The analysis of GC use will include all data captured during the 52-week randomized control period. For each subject, GC use is calculated as the cumulative GC dose taken for the purpose of disease control during the randomized control period. An analysis of covariance model with treatment indicator (Inebilizumab or placebo) and stratification factor as the explanatory variables is used. GC use for a purpose other than controlling IgG4-RD will not be included in the analysis. For subjects who discontinue prematurely from the randomized control period, further GC use is imputed as 0. Results is presented in terms of least square means (LSMEANS), treatment differences in LSMEANS, 95% CIs, and p-values.

### Exemplary Objectives

To evaluate the efficacy of Inebilizumab in reducing the risk of a disease flare in subjects with IgG4-RD. In embodiments, time to disease flare, defined as the time in days from Day 1 (dosing) to the date of the first treated and AC-determined IgG4 RD flare within the 52-week randomized control period. The date of disease flare is defined as the date of initiation of any flare treatment (new or increased GC treatment, other immunotherapy, or interventional procedure).

### Primary Objective

To evaluate the efficacy of Inebilizumab in reducing the risk of a disease flare in patients with IgG4-RD.

### Primary Endpoint

Time to disease flare, defined as the time in days from Day 1 (dosing) to the date of the first treated and AC-determined IgG4 RD flare within the 52-week randomized control period. The date of disease flare is defined as the date of initiation of any flare treatment (new or increased GC treatment, other immunotherapy, or interventional procedure), deemed necessary by the Investigator for the flare.).

### Secondary Objectives and Endpoints

### Secondary Objectives

a. To evaluate the safety and tolerability of Inebilizumab in patients with IgG4-RD.

b. To evaluate the effect of Inebilizumab on other measures of disease activity.

### Secondary Endpoints

a. Incidence of treatment emergent adverse events (TEAEs), treatment emergent serious adverse events (TESAEs), and treatment-emergent adverse events of special interest (AESIs) during the 52-week randomized control period and during the open label period.

b. The incidence of ADAs directed against Inebilizumab during the randomized control period.

c. Annualized flare rate for treated and AC-determined flares during the randomized control period.

d. Annualized flare rate for AC-determined flares, whether or not treated, during the randomized control period.

e. The proportion of subjects achieving flare-free complete remission at Week 52. Complete remission is defined as an IgG4-RD Responder Index score of 0 at Week 52, no AC-determined flare during the randomized control period, and no treatment for flare or disease control except the required 8-week GC taper.

f. Time to initiation of first treatment (medication or procedure) for new or worsening disease activity by the Investigator within the randomized control period, regardless of AC-determination of flare.

g. Glucocorticoid use calculated as the cumulative GC dose taken for the purpose of IgG4-RD disease control during the randomized control period.

### Exploratory Objectives and Endpoints

Table 7 lists the exploratory objectives and endpoints for this study.

TABLE 7
Exploratory Objectives and Endpoints

| Objective | Endpoint |
|---|---|
| To characterize the pharmacokinetics of | Serum concentration of inebilizumab and noncompartmental PK |
| inebilizumab in patients with IgG4-RD | parameters |
| To characterize the primary | Changes from baseline in peripheral B cell counts, including total |
| pharmacodynamic effect of inebilizumab in | B cells and B cell subsets |
| patients with IgG4-RD | |
| To explore the effect of inebilizumab on | The change from baseline to Week 52 in the Physician Global |
| other measures of disease activity. | Assessment of Disease Activity. |
| | Changes in patient functioning and health-related quality of life, as |
| | measured by changes from baseline to Week 52 in the following: |
| | SF-36v2 ® |
| | FACIT-fatigue |
| | Patient Global Assessment of Disease Activity |
| | Annualized flare rate for treated and AC-determined flares during |
| | the OLP |
| To assess the effect of inebilizumab on | The number of inpatient hospitalizations, days of hospitalization, days |
| health resource utilization in patients with | in an intensive care unit, emergency department visits, non-study |
| IgG4-RD | related physician visits, home-health visits (physician or nurse), |
| | disease-related imaging procedures, and disease-related |
| | procedures/surgeries (stenting, other). |
| To assess the effect of inebilizumab on | The change from baseline in immunoglobulin levels (IgG, IgG |
| circulating immunoglobulins | subclasses including IgG4, IgM, IgA, IgE, and total immunoglobulins). |
| To assess effect of inebilizumab on | Changes from baseline in serum levels of complement components C3 |
| complement components and the ELF score | and C4 and the ELF score. |
| To evaluate the effects of inebilizumab on | Changes from baseline in: |
| other disease-relevant measures, including | Blood gene expression profiles |
| other immune cell populations, biomarkers, | Serum biomarker expression (e.g., inflammation-related |
| and gene expression in patients with | cytokines/chemokines) |
| IgG4-RD | |
| To assess the relationship between genetic | Analysis of genetic alterations associated with disease activity and |
| variations, disease activity, and efficacy | response to treatment. |

AC = Adjudication Committee;
ELF = enhanced liver fibrosis;
FACIT-fatigue = Functional Assessment of Chronic Illness Therapy-Fatigue;
Ig = immunoglobulin;
OLP = open-label period;
PK = pharmacokinetic;
RCP = randomized-controlled period;
SF-36v2 = 36-item Short Form Health Survey version 2.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

### Numbered Embodiments

Notwithstanding the appended claims, the following numbered embodiments also form part of the instant disclosure.

Embodiment 1. A method of treating Immunoglobulin G4-related disease (IgG4-RD), comprising administering to a subject in need thereof an effective amount of Inebilizumab, wherein the administering is sufficient to reduce or eliminate an IgG4-RD flare, thereby treating the IgG4-RD.

Embodiment 2. The method of embodiment 1, wherein the administering is sufficient to reduce the IgG4-RD flare, as determined by: (a) reduced incidence of the IgG4-RD flare; (b) increased time to the IgG4-RD flare; or (c) a. and b.

Embodiment 3. The method of embodiment 2, comprising a., wherein the incidence of the IgG4-RD flare is reduced by at least about 5%, 10%, 20%, 30%, 40%, 50%, 60%, 70%, 80%, 90%, or 100% as compared to an otherwise comparable subject lacking the administering.

Embodiment 4. The method of embodiment 2, comprising b., wherein the time to IgG4-RD flare is increased by at least about 3 days, 5 days, 7 days, 10 days, 14 days, 20 days, 1 month, 2 months, 3 months, 4 months, 6 months, 8 months, 10 months, 1 year, or 1.5 years as compared to an otherwise comparable subject lacking the administering.

Embodiment 5. The method of any one of embodiments 2-4, comprising a. and b.

The method of any one of embodiments 1-5, wherein the subject in need thereof was previously administered a glucocorticoid.

Embodiment 7. The method of any one of embodiments 1-5, wherein the administering is effective in preventing new glucocorticoid administration in the subject in need thereof.

Embodiment 8. The method of any one of embodiments 1-6, wherein the administering is effective in preventing increased glucocorticoid administration in the subject in need thereof.

Embodiment 9. A method of treating Immunoglobulin G4-related disease (IgG4-RD), comprising administering to a subject in need thereof an effective amount of Inebilizumab, wherein the administering is sufficient to extend time to detection of a IgG4-RD flare by at least 3 days as compared to an otherwise comparable subject lacking the administering.

Embodiment 10. The method of embodiment 9, wherein the time to detection of the IgG4-RD flare is extended by at least about 5 days, 7 days, 10 days, 14 days, 20 days, 1 month, 2 months, 3 months, 4 months, 6 months, 8 months, 10 months, 1 year, or 1.5 years.

Embodiment 11. A method of reducing the incidence of a flare in a subject having Immunoglobulin G4-related disease (IgG4-RD), the method comprising administering to the subject an effective amount of an anti-CD19 binding agent, thereby reducing the incidence of the flare in the subject.

Embodiment 12. The method of embodiment 11, wherein the anti-CD19 binding agent is effective in reducing or eliminating at least one of CD19 positive: B cells, plasmablasts, plasma cells, or any combination thereof.

Embodiment 13. The method of any one of embodiments 11-12, wherein the anti-CD19 binding agent is an anti-CDI 9 antibody.

Embodiment 14. The method of embodiment 13, wherein the anti-CD19 antibody is Inebilizumab.

Embodiment 15. The method of any one of embodiments 1-14, wherein the administering is effective in preventing administration of: a secondary immunotherapy, a corticosteroid, or a steroid-sparing agent.

Embodiment 16. The method of embodiment 15, comprising the secondary immunotherapy, wherein the secondary immunotherapy comprises an antibody.

Embodiment 17. The method of embodiment 16, wherein the antibody is rituximab.

Embodiment 18. The method of embodiment 15, comprising the corticosteroid, wherein the corticosteroid comprises prednisone.

Embodiment 19. The method of embodiment 15, comprising the steroid-sparing agent, wherein the steroid-sparing agent is selected from the group consisting of: azathioprine and mycophenolate mofetil.

Embodiment 20. The method of any one of embodiments 1-19, wherein the IgG4-RD flare is determined by one or more of: an IgG4-RD symptom, a physical exam manifestation, a laboratory result, an imaging result, or a biopsy result.

Embodiment 21. The method of embodiment 20, comprising the IgG-RD symptom, wherein the IgG-RD symptom is selected from the group consisting of: headache, vision abnormality, salivary gland pain, lymph node swelling, pain, edema, dyspnea, cough, weight loss, itching, fatigue, rash, and combinations thereof.

Embodiment 22. The method of embodiment 21, comprising the vision abnormality, wherein the vision abnormality is selected from the group consisting of: diplopia, proptosis, foreign body sensation, eye pain, vision blurring, vision loss, scleritis, eye numbness, tearing, crusting, redness, lacrimal gland swelling, and combinations thereof.

Embodiment 23. The method of embodiment 20, comprising the physical exam manifestation, wherein the physical exam manifestation is selected from the group consisting of: central nervous system palsy, radiculomyelopathy, proptosis, supra-orbital swelling, peri-orbital swelling, field cuts, cranial nerve palsies, extraocular movement abnormality, infra-orbital/supra-orbital nerve enlargement, lacrimal gland swelling, salivary gland swelling, lymphadenopathy, increased respiratory rate, dry crackles, pleural effusion, palpable mass, edema, fibrosing mediastinitis, abdominal tenderness, jaundice, weight loss, abdominal tenderness, hyperpigmentation, erythematous nodules, and combinations thereof.

Embodiment 24. The method of embodiment 20, comprising the laboratory result, wherein the laboratory result is selected from the group consisting of: cerebrospinal fluid pleocytosis, cerebrospinal fluid increased protein, pituitary endocrine dysfunction, pulmonary function abnormality, elevated creatinine, decreased estimated glomerular filtration rate (eGFR), elevated bilirubin, elevated alkaline phosphatase, elevated gamma-glutamyl transferase, elevated amylase, elevated lipase, low fecal elastase, high glucose, elevated alanine aminotransferase, elevated aspartate transaminase, hematuria, proteinuria, and combinations thereof.

Embodiment 25. The method of embodiment 20, comprising the imaging result, wherein the imaging result is selected from the group consisting of: meningeal enhancement, pituitary mass, enlargement of extra-ocular muscle, enlargement of optic nerve, lacrimal gland swelling, salivary gland swelling, lymphadenopathy, pulmonary mass, pulmonary infiltrate, pulmonary fibrosis, pleural effusion, pleural thickening, peribronchovascular thickening, septal thickening, paravertebral mass, aneurysm, vascular thickening, ureteral stenosis, ureteral hydronephrosis, inflammation in retroperitoneum, inflammation in mesentery, inflammation in mediastinum, pancreatic mass, pancreatic enlargement, bile duct abnormality, pancreatic duct stricture, kidney enlargement, renal atrophy, pelvis thickening, and combinations thereof.

Embodiment 26. The method of embodiment 20, comprising the biopsy result, wherein the biopsy result is selected from the group consisting of: pachymeningeal flare, pituitary gland flare, orbital flare, lacrimal gland flare, salivary gland flare, lymph node flare, lung flare, blood vessel flare, retroperitoneum flare, mediastinum flare, mesentery flare, pancreatic flare, bile duct flare, kidney flare, skin disease flare, and combinations thereof.

Embodiment 27. The method of any one of embodiments 1-26, wherein from about 200-400 mg of the Inebilizumab is administered.

Embodiment 28. The method of embodiment 27, wherein from about 250-350 mg of the Inebilizumab is administered.

Embodiment 29. The method of any one of embodiments 27-28, wherein about 300 mg of the Inebilizumab is administered.

Embodiment 30. The method of any one of embodiments 1-29, wherein the administering is intravenous.

Embodiment 31. The method of any one of embodiments 1-30, comprising: a second administering of the Inebilizumab, a third administering of the Inebilizumab, or both a second and third administering of the Inebilizumab.

Embodiment 32. The method of embodiment 31, wherein each of the administering, the second administering, or the third administering are separated by at least about 2 weeks, 1 month, 2 months, 3 months, 4 months, 5 months, 6 months, 8 months, 10 months, or up to about 1 year.

Embodiment 33. A method of treating Immunoglobulin G4-related disease (IgG4-RD), comprising administering to a subject in need thereof: (a) a pharmaceutical composition that comprises a corticosteroid in an amount sufficient to reduce an immune response in the subject, and (b) a pharmaceutical composition that comprises Inebilizumab in an amount sufficient to reduce a level of at least one of CD19 positive B cells, plasmablasts, plasma cells, or any combination thereof in the subject in need thereof, thereby treating the IgG4-RD.

Embodiment 34. The method of embodiment 33, wherein the corticosteroid is a glucocorticoid.

Embodiment 35. The method of embodiment 34, wherein the glucocorticoid is selected from the group consisting of: prednisone, methylprednisolone, hydrocortisone, betamethasone, dexamethasone, and combinations thereof.

Embodiment 36. The method of embodiment 35, wherein the glucocorticoid is prednisone.

Embodiment 37. The method of any one of embodiments 33-36, wherein from about 1 mg/day to about 40 mg/day of the corticosteroid is administered.

Embodiment 38. The method of embodiment 37, wherein from about 5 mg/day to about 20 mg/day of the corticosteroid is administered.

Embodiment 39. The method of any one of embodiments 33-38, wherein the corticosteroid administering is tapered.

Embodiment 40. The method of embodiment 39, wherein the tapering comprises an administering of 20 mg/day, 15 mg/day, 10 mg/day, and 5 mg/day.

Embodiment 41. The method of any one of embodiments 39-40, wherein the tapering is completed over a period up to about 1 month, 2 months, 3 months, 4 months, or 5 months.

Embodiment 42. The method of embodiment 41, wherein the tapering is completed over a period of about 2 months.

Embodiment 43. The method of any one of embodiments 33-42, wherein the reduced immune response comprises at least a 1-fold decrease in: a level of leukocyte migration to a site of inflammation, capillary permeability, inflammation, and any combination thereof.

Embodiment 44. The method of any one of embodiments 33-43, wherein the Inebilizumab reduces the level of the at least one of CD19 positive B cells, plasmablasts, plasma cells, or any combination thereof by at least 1-fold, 5-fold, 10-fold, 30-fold, 60-fold, 90-fold, 150-fold, 200-fold, or 300-fold as compared to an otherwise comparable subject lacking the administering.

Embodiment 45. The method of any one of embodiments 39-44, wherein after the tapering, a reduction of IgG4-RD flare is observed as determined by: (a) reduced incidence of the IgG4-RD flare; (b) increased time to the IgG4-RD flare; or (c) a. and b.

Embodiment 46. The method of embodiment 45, comprising a., wherein the incidence of the IgG4-RD is reduced by at least about 10%, 20%, 30%, 40%, 50%, 60%, 70%, 80%, 90%, or 100% as compared to an otherwise comparable subject lacking the administering.

Embodiment 47. The method of embodiment 45, comprising b., w % herein the time to IgG4-RD flare is increased by at least about 3 days, 5 days, 7 days, 10 days, 14 days, 20 days, 1 month, 2 months, 3 months, 4 months, 6 months, 8 months, 10 months, 1 year, or 1.5 years as compared to an otherwise comparable subject lacking the administering.

Embodiment 48. The method of any one of embodiments 45-47, comprising a. and b.

Embodiment 49. The method of any one of embodiments 33-48, comprising determining a level of a marker in a sample of the subject in need thereof, wherein the marker is from a class selected from the group consisting of: immunoglobulin, complement, cellular, serum, RNA, DNA, and combinations thereof.

Embodiment 50. The method of any one of embodiments 45-49, wherein the IgG4-RD flare is determined by one or more of: an IgG4-RD symptom, a physical exam manifestation, a laboratory result, an imaging result, a biopsy result, or a combination thereof.

Embodiment 51. The method of embodiment 50, comprising the IgG-RD symptom, wherein the IgG-RD symptom is selected from the group consisting of: headache, vision abnormality, salivary gland pain, lymph node swelling, Dyspnea, pain, edema, dyspnea, cough, weight loss, itching, fatigue, rash, and combinations thereof.

Embodiment 52. The method of embodiment 51, comprising the vision abnormality, wherein the vision abnormality is selected from the group consisting of: Diplopia, proptosis, foreign body sensation, eye pain, vision blurring, vision loss, scleritis, eye numbness, tearing, crusting, redness, lacrimal gland swelling, and combinations thereof.

Embodiment 53. The method of embodiment 50, comprising the physical exam manifestation, wherein the physical exam manifestation is selected from the group consisting of: central nervous system palsy, radiculomyelopathy, proptosis, supra-orbital swelling, peri-orbital swelling, field cuts, cranial nerve palsies, extraocular movement abnormality, infra-orbital/supra-orbital nerve enlargement, lacrimal gland swelling, salivary gland swelling, lymphadenopathy, increased respiratory rate, dry crackles, pleural effusion, palpable mass, edema, fibrosing mediastinitis, abdominal tenderness, jaundice, weight loss, abdominal tenderness, hyperpigmentation, erythematous nodules, and combinations thereof.

Embodiment 54. The method of embodiment 50, comprising the laboratory result, wherein the laboratory result is selected from the group consisting of: cerebrospinal fluid pleocytosis, cerebrospinal fluid increased protein, pituitary endocrine dysfunction, pulmonary function abnormality, elevated creatinine, decreased estimated glomerular filtration rate (eGFR), elevated bilirubin, elevated alkaline phosphatase, elevated gamma-glutamyl transferase, elevated amylase, elevated lipase, low fecal elastase, high glucose, elevated alanine aminotransferase, elevated aspartate transaminase, hematuria, proteinuria, and combinations thereof.

Embodiment 55. The method of embodiment 50, comprising the imaging result, wherein the imaging result is selected from the group consisting of: meningeal enhancement, pituitary mass, enlargement of extra-ocular muscle, enlargement of optic nerve, lacrimal gland swelling, salivary gland swelling, lymphadenopathy, pulmonary mass, pulmonary infiltrate, pulmonary fibrosis, pleural effusion, pleural thickening, peribronchovascular thickening, septal thickening, paravertebral mass, aneurysm, vascular thickening, ureteral stenosis, ureteral hydronephrosis, inflammation in retroperitoneum, inflammation in mesentery, inflammation in mediastinum, pancreatic mass, pancreatic enlargement, bile duct abnormality, pancreatic duct stricture, kidney enlargement, renal atrophy, pelvis thickening, and combinations thereof.

Embodiment 56. The method of embodiment 50, comprising the biopsy result, wherein the biopsy result is selected from the group consisting of: pachymeningeal flare, pituitary gland flare, orbital flare, lacrimal gland flare, salivary gland flare, lymph node flare, lung flare, blood vessel flare, retroperitoneum flare, mediastinum flare, mesentery flare, pancreatic flare, bile duct flare, kidney flare, skin disease flare, and combinations thereof.

Embodiment 57. The method of any one of embodiments 33-56, comprising administering an antihistamine, an antipyretic, or both.

Embodiment 58. The method of embodiment 57, comprising the antihistamine, wherein the antihistamine is diphenhydramine.

Embodiment 59. The method of embodiment 57, comprising the antipyretic, wherein the antipyretic is acetaminophen.

Embodiment 60. The method of any one of embodiments 57-59, wherein the antihistamine, antipyretic, or both are administered prior to the pharmaceutical composition that comprises Inebilizumab.

Embodiment 61. A method of treating Immunoglobulin G4-related disease (IgG4-RD), the method comprising: administering Inebilizumab to a patient in need of treatment for IgG4-RD, wherein the Inebilizumab is administered intravenously at a dose of 300 mg every 6 months.

Embodiment 62. The method of embodiment 61, wherein two weeks prior to administering the 300 mg Inebilizumab every 6 months, an initial 300 mg Inebilizumab dose is administered to the subject.

Embodiment 63. A method of treating a patient diagnosed with Immunoglobulin G4-related disease (IgG4-RD), the method comprising: administering Inebilizumab to a patient diagnosed with IgG4-RD, wherein the Inebilizumab is administered intravenously with a first initial dose of 300 mg, a second initial dose of 300 mg two weeks after the first initial dose, and subsequent doses of 300 mg every 6 months following the first initial dose.

Embodiment 64. The method of any one of embodiments 29-32, wherein two weeks prior to administering the 300 mg Inebilizumab every 6 months, an initial 300 mg Inebilizumab dose is administered to the subject.

1. A method of treating Immunoglobulin G4-related disease (IgG4-RD), comprising administering to a subject in need thereof an effective amount of Inebilizumab, wherein the Inebilizumab comprises a variable heavy chain comprising SEO ID NO: 1 and a variable light chain comprising SEQ ID NO: 5.

2. The method of claim 1, wherein the administering is sufficient to reduce the IgG4-RD flare, as determined by:
a. reduced incidence of the IgG4-RD flare;
b. increased time to the IgG4-RD flare; or
c. a. and b.

3. The method of claim 2, comprising a., wherein the incidence of the IgG4-RD flare is reduced by at least about 5% as compared to an otherwise comparable subject lacking the administering.

4. The method of claim 2, comprising b., wherein the time to IgG4-RD flare is increased by at least about 3 days as compared to an otherwise comparable subject lacking the administering.

5. (canceled)

6. The method of claim 1, wherein the subject in need thereof was previously administered a glucocorticoid.

7. The method of claim 1, wherein the administering is effective in preventing new glucocorticoid administration in the subject in need thereof.

8.-17. (canceled)

18. The method of claim 1, comprising administering a corticosteroid.

19. The method of claim 1, comprising administering a steroid-sparing agent, wherein the steroid-sparing agent is selected from the group consisting of: azathioprine and mycophenolate mofetil.

20.-26. (canceled)

27. The method of claim 1, wherein from about 200-400 mg of the Inebilizumab is administered.

28. (canceled)

29. The method of claim 27, wherein about 300 mg of the Inebilizumab is administered.

30. The method of claim 29, wherein the 300 mg is administered every 6 months.

31. The method of claim 30, comprising a first dose of 300 mg, a second dose of 300 mg two weeks after the first dose, and subsequent doses of 300 mg every 6 months following the first initial dose.

32. (canceled)

33. A method of treating Immunoglobulin G4-related disease (IgG4-RD), comprising administering to a subject in need thereof:
a. a pharmaceutical composition that comprises a corticosteroid in an amount sufficient to reduce an immune response in the subject; and
b. a pharmaceutical composition that comprises Inebilizumab in an amount sufficient to reduce a level of at least one of CD19 positive B cells, plasmablasts, plasma cells, or any combination thereof in the subject in need thereof, wherein the Inebilizumab comprises a variable heavy chain comprising SEO ID NO: 1 and a variable light chain comprising SEO ID NO: 5.

34. The method of claim 33, wherein the corticosteroid is a glucocorticoid.

35. The method of claim 34, wherein the glucocorticoid is selected from the group consisting of: prednisone, methylprednisolone, hydrocortisone, betamethasone, dexamethasone, and combinations thereof.

36. The method of claim 35, wherein the glucocorticoid is prednisone.

37. The method of claim 33, wherein from about 1 mg/day to about 40 mg/day of the corticosteroid is administered.

38-43. (canceled)

44. The method of claim 33, wherein the Inebilizumab reduces the level of the at least one of CD19 positive B cells, plasmablasts, plasma cells, or any combination thereof by at least 1-fold as compared to an otherwise comparable subject lacking the administering.

45. The method of claim 33, wherein the corticosteroid is tapered, and wherein after the tapering, a reduction of IgG4-RD flare is observed as determined by:
a. reduced incidence of the IgG4-RD flare;
b. increased time to the IgG4-RD flare; or
c. a. and b.

46.-48. (canceled)

49. The method of claim 33, comprising determining a level of a marker in a sample of the subject in need thereof, wherein the marker is from a class selected from the group consisting of: immunoglobulin, complement, cellular, serum, RNA, DNA, and combinations thereof.

50.-56. (canceled)

57. The method of claim 33, comprising administering an antihistamine, an antipyretic, or both.

58.-59. (canceled)

60. The method of claim 57, wherein the antihistamine, antipyretic, or both are administered prior to the pharmaceutical composition that comprises Inebilizumab.

61.-62. (canceled)

63. A method of treating a patient with Immunoglobulin G4-related disease (IgG4-RD), the method comprising: administering Inebilizumab to a patient diagnosed with IgG4-RD, wherein the Inebilizumab comprises a variable heavy chain comprising SEQ ID NO. 1 and a variable light chain comprising SEQ ID NO: 5 and is administered intravenously with a first initial dose of 300 mg, a second initial dose of 300 mg two weeks after the first initial dose, and subsequent doses of 300 mg every 6 months following the first initial dose.

64. (canceled)

65. The method of claim 29, wherein two weeks prior to administering the 300 mg Inebilizumab every 6 months, an initial 300 mg Inebilizumab dose is administered to the subject.

66. The method of claim 1, wherein the Inebilizumab comprises a heavy chain comprising SEQ ID NO: 9 and a light chain comprising SEQ ID NO: 10.

67. The method of claim 33, wherein the Inebilizumab comprises a heavy chain comprising SEQ ID NO: 9 and a light chain comprising SEQ ID NO: 10.

68. The method of claim 63, wherein the Inebilizumab comprises a heavy chain comprising SEQ ID NO: 9 and a light chain comprising SEQ ID NO: 10.
